SEQUENCE LISTING (5 sequences)

SEQ ID NO: 1 - protein, 708 aa, Bos taurus
MKLFVPALLSLGALGLCLAAPRKNVRWCTISQPEWFKCRRWQWRMKKLGAPSITCVRRAFALECIRAIAEKKADAVTLDGGMVFEAGRDPYKLRPVAAEIYGTKESPQTHYYAVAVVKKGSNFQLDQLQGRKSCHTGLGRSAGWIIPMGILRPYLSWTESLEPLQGAVAKFFSASCVPCIDRQAYPNLCQLCKGEGENQCACSSREPYFGYSGAFKCLQDGAGDVAFVKETTVFENLPEKADRDQYELLCLNNSRAPVDAFKECHLAQVPSHAVVARSVDGKEDLIWKLLSKAQEKFGKNKSRSFQLFGSPPGQRDLLFKDSALGFLRIPSKVDSALYLGSRYLTTLKNLRETAEEVKARYTRVVWCAVGPEEQKKCQQWSQQSGQNVTCATASTTDDCIVLVLKGEADALNLDGGYIYTAGKCGLVPVLAENRKSSKHSSLDCVLRPTEGYLAVAVVKKANEGLTWNSLKDKKSCHTAVDRTAGWNIPMGLIVNQTGSCAFDEFFSQSCAPGADPKSRLCALCAGDDQGLDKCVPNSKEKYYGYTGAFRCLAEDVGDVAFVKNDTVWENTNGESTADWAKNLNREDFRLLCLDGTRKPVTEAQSCHLAVAPNHAVVSRSDRAAHVKQVLLHQQALFGKNGKNCPDKFCLFKSETKNLLFNDNTECLAKLGGRPTYEEYLGTEYVTAIANLKKCSTSPLLEACAFLTR

SEQ ID NO: 2 - protein, 710 aa, Homo sapiens
MKLVFLVLLFLGALGLCLAGRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPIGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTAGKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 3 - protein, 142 aa, Bos taurus
MRFFVPLFLVGILFPAILAKQFTKCELSQLLKDIDGYGGIALPELICTMFHTSGYDTQAIVENNESTEYGLFQISNKLWCKSSQVPQSRNICDISCDKFLDDDITDDIMCAKKILDIKGIDYWLAHKALCTEKLEQWLCEKL

SEQ ID NO: 4 - protein, 147 aa, Bos taurus
MKALVILGFLFLSVAVQGKVFERCELARTLKKLGLDGYKGVSLANWLCLTKWESSYNTKATNYNPSSESTDYGIFQINSKWWCNDGKTPNAVDGCHVSCRELMENDIAKAVACAKHIVSEQGITAWVAWKSHCRDHDVSSYVEGCTL

SEQ ID NO: 5 - protein, 278 aa, Bos taurus
MRIAVICFCLLGIASALPVKPTSSGSSEEKQLNNKYPDAVATWLKPDPSQKQTFLAPQNSVSSEETDDNKQNTLPSKSNESPEQTDDLDDDDDNSQDVNSNDSDDAETTDDPDHSDESHHSDESDEVDFPTDIPTIAVFTPFIPTESANDGRGDSVAYGLKSRSKKFRRSNVQSPDATEEDFTSHIESEEMHDAPKKTSQLTDHSKETNSSELSKELTPKAKDKNKHSNLIESQENSKLSQEFHSLEDKLDLDHKSEEDKHLKIRISHELDSASSEVN

(19) United States
(12) Patent Application Publication
(10) Pub. No.: US 2025/0295588 A1
(43) Pub. Date: Sep. 25, 2025

(54) PROTEIN AND BIOPOLYMER COMPLEXES AND METHODS OF MAKING AND USING THE SAME

(71) Applicants: Dairy Management Inc., Rosemont, IL (US); Cornell University, Ithaca, NY (US)

(72) Inventors: Tiantian Lin, Ithaca, NY (US); Younas Dadmohammadi, Ithaca, NY (US); Alireza Abbaspourrad, Ithaca, NY (US); Yufeng Zhou, Ithaca, NY (US); Hongmin Dong, Ithaca, NY (US); Gopinathan H. Meletharayil, Palatine, IL (US); Emil S. Nashed, North Haledon, NJ (US); Rohit Kapoor, Elk Grove Village, IL (US)

(21) Appl. No.: 18/865,705

(22) PCT Filed: May 16, 2023
(86) PCT No.: PCT/US2023/067034

§ 371 Date: Nov. 14, 2024

Related U.S. Application Data
(60) Provisional application No. 63/342,287, filed on May 16, 2022.

(51) Int. Cl. A61K 9/14; A61K 38/40; A61P 31/04

(52) U.S. Cl. CPC A61K 9/146; A61K 38/40; A61P 31/04

(57) ABSTRACT

Described herein are complexes including a protein, an anionic biopolymer, and a cationic biopolymer along with methods of making and using such complexes. The complex may include lactoferrin. gelatin, and a polysaccharide.

### STATEMENT REGARDING ELECTRONIC FILING OF A SEQUENCE LISTING

A Sequence Listing in XML text format, entitled 1213-3WO_ST26.xml, 7,184 bytes in size, generated on Apr. 25, 2023, and filed herewith, is hereby incorporated by reference into the specification for its disclosures.

### FIELD

This invention relates to complexes including a protein, an anionic biopolymer, and a cationic biopolymer and to methods of making and using such complexes.

### BACKGROUND

Lactoferrin (LF) is an iron-binding multifunctional protein occurring in many biological secretions, including milk. It possesses iron binding/transferring, antibacterial, antiviral, anti-inflammatory, and anti-carcinogenic properties. It promotes cell growth and detoxifies harmful free radicals and has anti-bacterial, anti-viral, anti-inflammatory, and anti-carcinogenic properties. Because of the multiple biological functions of LF, it has been incorporated into many commercialized products, including infant formulas, nutritional supplements, therapeutic drinks, and cosmetics. However, LF is sensitive to denaturation induced by thermal processing, especially under neutral pH conditions, which causes structural changes and the loss of biological functionality.

### SUMMARY OF THE INVENTION

A first aspect of the present invention is directed to a complex comprising: a protein; an anionic biopolymer; and a cationic biopolymer; wherein the protein, anionic biopolymer, and cationic biopolymer are associated via electrostatic interactions.

A second aspect of the present invention is directed to a composition comprising a complex of the present invention. In some embodiments, the composition is an aqueous composition.

A further aspect of the present invention is directed to a method of preparing a complex, the method comprising: providing a composition comprising a protein, an anionic biopolymer, and a cationic biopolymer at a pH in a range of about 3, 3.5, or 4 to about 4.5 or 5; and mixing the composition, thereby providing the complex.

A further aspect of the present invention is directed to an article comprising a complex of the present invention and/or a composition of the present invention. In some embodiments, the article is a food product (e.g., infant formula, a dairy product, etc.), nutritional supplement, therapeutic drink, and/or cosmetic.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 shows schematics depicting exemplary methods for preparing a ternary complex of the present invention using branched polysaccharides (FIG. 1, panel a) or linear polysaccharides (FIG. 1, panel b) according to some embodiments of the present invention.

FIG. 2 shows plots of turbidity for mixtures including lactoferrin (LF), gelatin (G), and a polysaccharide (PS) selected from gum arabic (GA), soy soluble polysaccharides (SSP), high methyl pectin (HMP), kappa carrageenan (Kappa), and iota carrageenan (Iota) as a function of compositions in mass ratios at a total concentration of 0.2% at pH 4.

FIG. 3 shows plots of turbidity for PS (GA/SSP)-LF-G mixtures as a function of compositions in mass ratios at a total concentration of 1% at pH 4.

FIG. 4 shows graphs of zeta potential of PS (GA/SSP/HMP/Kappa/lota)-LF-G mixtures as a function of compositions in mass ratios at a total concentration of 1% at pH 4.

FIG. 5 shows a graph of turbidity of PS (GA/SSP/HMP/Kappa/Iota)-LF-G mixtures at pH 4 as a function of salt concentration (0-500 mM).

FIG. 6 shows optical microscopy images of PS (GA/SSP/HMP/Kappa/Iota)-LF-G mixtures.

FIG. 7 shows confocal microscopy images of PS (GA/HMP/Iota)-LF-G mixtures with LF labeled with FTIC (light gray).

FIG. 8 shows a graph of turbidity for unheated and heated (75° C./2 min or 90° C./2 min) LF and re-dispersed ternary complexes in PBS (10 mM, pH 7).

FIG. 9 shows a graph of mean particle size for unheated and heated (75° C./2 min or 90° C./2 min) LF and re-dispersed ternary complexes in PBS (10 mM, pH 7).

FIG. 10 shows an image of an SDS-PAGE gel with unheated and heated (75° C./2 min or 90° C./2 min) LF and re-dispersed ternary complexes in PBS (10 mM, pH 7).

FIG. 11 shows an image of another SDS-PAGE gel with unheated and heated (75° C./2 min or 90° C./2 min) LF and re-dispersed ternary complexes in PBS (10 mM, pH 7).

FIG. 12 shows graphs of circular dichroism spectroscopy for unheated and heated (75° C./2 min or 90° C./2 min) LF and re-dispersed ternary complexes in PBS (10 mM, pH 7).

FIG. 13 shows graphs of intrinsic fluorescence for unheated and heated (75° C./2 min) LF and re-dispersed ternary complexes in PBS (10 mM, pH 7).

FIG. 14 shows a graph of the change in peak fluorescence for unheated and heated (75° C./2 min) LF and re-dispersed ternary complexes in PBS (10 mM, pH 7).

FIG. 15 shows the growth of Staphylococcus aureus as indicated by OD625 with incubation at 37° C. with LF at a series of concentrations (w/v %) for 0 h, 24 h, or 48 h (Panel A of FIG. 15) and incubation at 37° C. with LF (0.1 w/v %) and re-dispersed ternary complexes (0.2 w/v %) in PBS (10 mM, pH 7) before thermal treatment (Panel B of FIG. 15), after thermal treatment at 75° C./2 min (Panel C of FIG. 15), and after thermal treatment at 90° C./2 min (Panel D of FIG. 15). The pairwise comparison between each sample and control was performed and the significance difference level was shown as * (p≤0.05), ** (p≤0.01), (p≤0.001), or **** (p≤0.0001).

FIG. 16 shows the growth of Escherichia coli as indicated by OD625 with incubation at 37° C. with LF at a series of concentrations (w/v %) for 0 h, 12 h, or 24 h (Panel A of FIG. 16) and incubation at 37° C. with LF (0.1 w/v %) and re-dispersed ternary complex (0.2 w/v %) in PBS (10 mM, pH 7) before thermal treatment (Panel B of FIG. 16), after thermal treatment at 75° C./2 min (Panel C of FIG. 16), and after thermal treatment at 90° C./2 min (Panel D of FIG. 16). The pairwise comparison between each sample and control was performed and the significance difference level was shown as * (p≤0.05), ** (p≤0.01), *** (p≤0.001), or **** (p≤0.0001).

FIG. 17 shows scanning electron microscopy images of PS (GA/SSP/HMP/Kappa/Iota)-LF-G complex under a scale of 2 μm (Panels A1-E1 of FIG. 17) and 200 nm (Panels A2-B2 of FIG. 17).

FIG. 18 shows graphs of the turbidity (Panel A of FIG. 18) and mean particle size (Panel B of FIG. 18) of LF (0.1 w/v %) and re-dispersed ternary complexes (0.2 w/v %) in PBS (10 mM, pH 7) after oil bath heating (145° C.) for 0 s, 2 s, 10 s, 30 s, and 60 s.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B, and C, it is specifically intended that any of A, B, or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

A “portion” or “fragment” of a nucleotide sequence or polypeptide (including a domain) will be understood to mean a nucleotide sequence or polypeptide of reduced length (e.g., reduced by 1, 2, 3, 4, 5, 6, 7, 8, 9, 10, 11, 12, 13, 14, 15, 16, 17, 18, 19, 20 or more residue(s) (e.g., nucleotide(s) or peptide(s)) relative to a reference nucleotide sequence or polypeptide, respectively, and comprising, consisting essentially of and/or consisting of a nucleotide sequence or polypeptide of contiguous residues, respectively, identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleotide sequence or polypeptide.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence as compared to a reference polypeptide.

As used herein, the phrase “substantially identical,” or “substantial identity” in the context of two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5% or 100% nucleotide or amino acid residue identity, when compared and aligned for maximum correspondence, as measured using one of the following sequence comparison algorithms or by visual inspection. In some embodiments of the invention, the substantial identity exists over a region of consecutive nucleotides of a nucleotide sequence of the invention that is about 10 nucleotides to about 20 nucleotides, about 10 nucleotides to about 25 nucleotides, about 10 nucleotides to about 30 nucleotides, about 15 nucleotides to about 25 nucleotides, about 30 nucleotides to about 40 nucleotides, about 50 nucleotides to about 60 nucleotides, about 70 nucleotides to about 80 nucleotides, about 90 nucleotides to about 100 nucleotides, or more nucleotides in length, and any range therein, up to the full length of the sequence. In some embodiments, the nucleotide sequences can be substantially identical over at least about 20 nucleotides (e.g., about 20, 21, 22, 23, 24, 25, 26, 27, 28, 29, 30, 31, 32, 33, 34, 35, 36, 37, 38, 39, 40 nucleotides). In some embodiments, a substantially identical nucleotide or protein sequence performs substantially the same function as the nucleotide (or encoded protein sequence) to which it is substantially identical.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, e.g., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Provided according to embodiments of the present invention are complexes comprising a protein, an anionic biopolymer, and a cationic biopolymer. A complex of the present invention may be a ternary complex in that the complex comprises three different components such as a protein, anionic biopolymer, and cationic biopolymer that are each different from each other (e.g., different in chemical structure). In some embodiments, a complex of the present invention comprises at least three different components, such as a protein, anionic biopolymer, and cationic biopolymer, that are each different from each other (e.g., different in chemical structure).

A complex of the present invention may comprise one or more protein(s), one or more anionic biopolymer(s), and one or more cationic biopolymer(s), which may be associated with one another via electrostatic interactions. A complex of the present invention may have a zeta potential of about −5, −4, −3, −2, −1, or 0 mV to about +1, +2, +3, +4, or +5 mV, optionally when present in a composition (e.g., water and/or a buffer) having a pH of about 3, 3.5, or 4 to about 4.5, 5, 5.5, 6, 6.5, 7, or 7.5. In some embodiments, a complex of the present invention has a zeta potential of about −5, −4, −3, −2, −1, 0, +1, +2, +3, +4, or +5 mV, optionally when present in a composition (e.g., water and/or a buffer) having a pH of about 3, 3.5, or 4 to about 4.5, 5, 5.5, 6, 6.5, 7, or 7.5. In some embodiments, a complex of the present invention has a zeta potential of about 0 mV, optionally when present in a composition (e.g., water and/or a buffer) having a pH of about 3, 3.5, or 4 to about 4.5, 5, 5.5, 6, 6.5, 7, or 7.5. A complex of the present invention may have a net negative charge at a pH of about 6.5 to about 7.5, optionally a net negative charge at a pH of about 6.5, 7, or 7.5.

A complex of the present invention may comprises a protein, an anionic biopolymer, and/or a cationic biopolymer in an amount of about 1%, 5%, 10%, 15%, 20%, 25%, 30%, 35%, 40%, 45%, 50%, 55%, 60%, 65%, 70%, 75%, 80%, or 85% w/w of the complex or more. In some embodiments, a protein, an anionic biopolymer, and/or a cationic biopolymer is present in a complex of the present invention in an amount of about 1%, 5%, 10%, 15%, 20%, 25%, 30%, 35%, 40%, 45%, 50%, 55%, 60%, 65%, 70%, 75%, 80%, or 85% w/w of the complex. In some embodiments, a complex of the present invention comprises a protein in an amount of about 30%, 35%, or 40% to about 45%, 50%, 55%, 60%, 65%, 70%, 75%, 80%, or 85% w/w, a cationic biopolymer in an amount of about 1%, 5%, or 10% to about 15%, 20%, 25%, 30%, 35%, or 40% by w/w, and an anionic biopolymer in an amount of about 1%, 5%, or 10% to about 15%, 20%, 25%, 30%, 35%, or 40% w/w. In some embodiments, the complex comprises a protein in an amount of about 40% to about 75% w/w, a cationic biopolymer in an amount of about 10% to about 30% by w/w, and an anionic biopolymer in an amount of about 10% to about 30% w/w.

One or more (e.g., 1, 2, 3, 4, 5, 10, 15, 20, 30, 40, or 50, or more) protein(s) may be present in a complex of the present invention. In some embodiments, a complex of the present invention comprises two or more proteins that may be the same or different from each other. In some embodiments, a complex of the present invention comprises one or more protein molecule(s) (e.g., individual proteins and/or protein monomers) that are the same. A protein present in a complex of the present invention and/or used to prepare a complex of the present invention may have a net positive charge, optionally at a pH of about 3, 3.5, or 4 to about 4.5, 5, 6, 7, or 8. In some embodiments, a protein present in a complex of the present invention and/or used to prepare a complex of the present invention, optionally when present in a composition (e.g., water and/or a buffer) having a pH of about 3, 3.5, or 4 to about 4.5 or 5, may have a zeta potential of greater than about +10 mV such as a zeta potential of about +11, +12, +13, +14, +15, +16, +17, +18, +19, +20, +21, +22, +23, +24, +25, +26, +27, +28, +29, or +30 mV or more.

A protein present in a complex of the present invention and/or used to prepare a complex of the present invention may have a globular structure. In some embodiments, a protein present in a complex of the present invention and/or used to prepare a complex of the present invention is soluble in water at a pH of less than about 8 such as a pH of about 3, 3.5, 4, 4.5, 5, 5.5, 6, 6.5, 7, 7.5, or 8, optionally a solubility of about 5, 10, 15, or 20 mg/L or more in water at a pH of less than about 8 and at about 25° C. In some embodiments, a protein present in a complex of the present invention and/or used to prepare a complex of the present invention is a dairy protein. A “dairy protein” as used herein refers to a protein that is found naturally in a dairy product and/or milk and/or that is derived from such a naturally occurring protein to have an amino acid sequence having at least 70% sequence identity to the naturally occurring protein's amino acid sequence. For example, in some embodiments, a dairy protein is naturally found in a milk (e.g., an animal milk) and/or the protein is isolated from a milk, or the protein is synthetically prepared to have an amino acid sequence having at least 70% sequence identity to the naturally occurring protein's amino acid sequence.

Exemplary proteins that may be present in a complex of the present invention and/or used to prepare a complex of the present invention include, but are not limited to, lactoferrin, alpha lactalbumin, lysozyme, and/or osteopontin. In some embodiments, a complex of the present invention comprises lactoferrin. A protein of the present invention may be from any source (e.g., plant, animal, etc.). In some embodiments, the protein is obtained and/or derived from an animal such as a mammal (e.g., a bovine, goat, sheep, or human). In some embodiments, a protein present in a complex of the present invention has an amino acid sequence having about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to one or more of SEQ ID NOs: 1-5. In some embodiments, a protein present in a complex of the present invention has an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to one or more of SEQ ID NOs: 1-5. In some embodiments, a protein present in a complex of the present invention has an amino acid sequence having about 100% sequence identity to one or more of SEQ ID NOs: 1-5.

One or more (e.g., 1, 2, 3, 4, 5, 10, 15, 20, 30, 40, or 50, or more) cationic biopolymer(s) may be present in a complex of the present invention. In some embodiments, a complex of the present invention comprises two or more cationic biopolymers that may be the same or different from each other. In some embodiments, a complex of the present invention comprises one or more cationic biopolymer(s) molecule(s) (e.g., individual biopolymer compounds) that are the same. A cationic biopolymer present in a complex of the present invention and/or used to prepare a complex of the present invention may have a net positive charge, optionally at a pH of about 3, 3.5, or 4 to about 4.5, 5, 6, 7, or 8. In some embodiments, a cationic biopolymer present in a complex of the present invention and/or used to prepare a complex of the present invention, optionally when present in a composition (e.g., water and/or a buffer) having a pH of about 3, 3.5, or 4 to about 4.5 or 5, may have a zeta potential of greater than about +10 m V such as a zeta potential of about +11, +12, +13, +14, +15, +16, +17, +18, +19, +20, +21, +22, +23, +24, +25, +26, +27, +28, +29, or +30 mV or more. A cationic biopolymer present in a complex of the present invention and/or used to prepare a complex of the present invention may have a pI and/or a pKa of about 7 or more such as about 7, 7.5, 8, 8.5, 9, 9.5, 10, 10.5, 11, or more.

A “cationic biopolymer” as used herein refers to a polymer that carries or can carry a positive charge and that is produced by a living organism or is a derivative thereof and/or is synthetically prepared to have a structure consistent with a polymer produced by a living organism or a derivative thereof. In some embodiments, a cationic biopolymer has at least one free amine and/or hydroxyl group present on a majority of the monomeric units of the polymer. In some embodiments, a free amine and/or hydroxyl group may be present on each of the monomeric units of the polymer backbone. Exemplary cationic biopolymers include, but are not limited to, proteins, polyamino acids, and/or polysaccharides that can include a positive charge (optionally have a net positive charge). As one of ordinary skill in the art will understand, a cationic biopolymer may be synthetically obtained (e.g., through laboratory synthesis) and/or obtained and/or derived from nature (e.g., from a living or previously living organism). Therefore, a cationic biopolymer may be the same as a polymer found in nature (i.e., a native cationic biopolymer) or may be a derivative thereof. For example, a cationic biopolymer of the present invention may be a derivative of a polymer produced by a living organism, the derivative caused by the synthetic method used to obtain or isolate the biopolymer from nature. In some embodiments, a cationic biopolymer may be a polymer produced by bacteria and/or microbes. Exemplary cationic biopolymers that may be present in a complex of the present invention and/or used to prepare a complex of the present invention include, but are not limited to, gelatin, chitosan, lysozyme, and/or a polyamino acid. In some embodiments, a complex of the present invention comprises a gelatin. The cationic biopolymer may be a biopolymer found naturally in an animal, plant, and/or fungus and/or may be derived from such a naturally occurring biopolymer. In some embodiments, a cationic biopolymer is a biopolymer naturally found in an animal, plant, and/or fungus and is isolated therefrom. In some embodiments, a cationic biopolymer is synthetically prepared based on a biopolymer naturally found in an animal, plant, and/or fungus. In some embodiments, a cationic biopolymer is obtained from a source (e.g., an animal, plant, and/or fungus) and/or synthetically prepared based on a natural biopolymer and the obtained and/or prepared biopolymer is modified (e.g., modified to have a cationic functional group, etc.). A cationic biopolymer of the present invention may be from any source (e.g., plant, animal, etc.). In some embodiments, the cationic biopolymer is obtained and/or derived from an animal such as a mammal (e.g., a bovine, goat, sheep, or human).

One or more (e.g., 1, 2, 3, 4, 5, 10, 15, 20, 30, 40, or 50, or more) anionic biopolymer(s) may be present in a complex of the present invention. In some embodiments, a complex of the present invention comprises two or more anionic biopolymers that may be the same or different from each other. In some embodiments, a complex of the present invention comprises one or more anionic biopolymer(s) molecule(s) (e.g., individual biopolymer compounds) that are the same. An anionic biopolymer present in a complex of the present invention and/or used to prepare a complex of the present invention may have a net negative charge, optionally at a pH of about 3, 3.5, or 4 to about 4.5, 5, 6, 7, or 8. In some embodiments, an anionic biopolymer present in a complex of the present invention and/or used to prepare a complex of the present invention, optionally when present in a composition (e.g., water and/or a buffer) having a pH of about 3, 3.5, or 4 to about 4.5 or 5, may have a zeta potential of less than about −10 mV such as a zeta potential of about −11, −12, −13, −14, −15, −16, −17, −18, −19, −20, −21, −22, −23, −24, −25, −26, −27, −28, −29, −30, −31, −32, −33, −34, −35, −36, −37, −38, −39, −40, −41, −42, −43, −44, −45, −46, −47, −48, −49, −50, −51, −52, −53, −54, or −55 mV or more. In some embodiments, an anionic biopolymer present in a complex of the present invention and/or used to prepare a complex of the present invention, optionally when present in a composition (e.g., water and/or a buffer) having a pH of about 3, 3.5, or 4 to about 4.5 or 5, has a zeta potential in a range of about −10 mV to about −25 mV. In some embodiments, an anionic biopolymer present in a complex of the present invention and/or used to prepare a complex of the present invention, optionally when present in a composition (e.g., water and/or a buffer) having a pH of about 3, 3.5, or 4 to about 4.5 or 5, has a zeta potential in a range of about −35 mV to about −55 mV. An anionic biopolymer present in a complex of the present invention and/or used to prepare a complex of the present invention may have a pKa of about 4 or less such as a pKa of about 4, 3.5, 3, 2.5, 2, 1.5, or 1 or less.

An “anionic biopolymer” as used herein refers to a polymer that carries or can carry a negative charge and that is produced by a living organism or is a derivative thereof and/or is synthetically prepared to have a structure consistent with a polymer produced by a living organism or a derivative thereof. In some embodiments, an anionic biopolymer has at least one free amine and/or hydroxyl group present on a majority of the monomeric units of the polymer. In some embodiments, a free amine and/or hydroxyl group may be present on each of the monomeric units of the polymer backbone. Exemplary anionic biopolymers include, but are not limited to, proteins, polyamino acids, glycosaminoglycans, glycoproteins, and/or polysaccharides that can include a negative charge (optionally have a net negative charge). As one of ordinary skill in the art will understand, an anionic biopolymer may be synthetically obtained (e.g., through laboratory synthesis) and/or obtained and/or derived from nature (e.g., from a living or previously living organism). Therefore, an anionic biopolymer may be the same as a polymer found in nature (i.e., a native anionic biopolymer) or may be a derivative thereof. For example, an anionic biopolymer of the present invention may be a derivative of a polymer produced by a living organism, the derivative caused by the synthetic method used to obtain or isolate the biopolymer from nature. In some embodiments, an anionic biopolymer may be a polymer produced by bacteria and/or microbes. In some embodiments, an anionic biopolymer present in a complex of the present invention and/or used to prepare a complex of the present invention is a polysaccharide, a glycosaminoglycan, a glycoprotein, and/or a polyamino acid. In some embodiments, a complex of the present invention comprises a polysaccharide. In some embodiments, a complex of the present invention comprises a linear polysaccharide (i.e., a polysaccharide that is a straight chain of linked/attached monosaccharides, optionally wherein the monosaccharides are each linked by an α-1,4-glycosidic bond or an β-1,4-glycosidic bond). In some embodiments, a complex of the present invention comprises a branched polysaccharide (e.g., a polysaccharide including two or more monosaccharides that are linked by an α-1,4-glycosidic bond and two or more monosaccharides that are linked by an α-1,6-glycosidic bond). The anionic biopolymer may be a biopolymer found naturally in an animal, plant, and/or fungus and/or may be derived from such a naturally occurring biopolymer. In some embodiments, an anionic biopolymer is a biopolymer naturally found in an animal, plant, and/or fungus and is isolated therefrom. In some embodiments, an anionic biopolymer is synthetically prepared based on a biopolymer naturally found in an animal, plant, and/or fungus. In some embodiments, an anionic biopolymer is obtained from a source (e.g., an animal, plant, and/or fungus) and/or synthetically prepared based on a natural biopolymer and the obtained and/or prepared biopolymer is modified (e.g., modified to have an anionic functional group, etc.). An anionic biopolymer of the present invention may be from any source (e.g., plant, animal, etc.). In some embodiments, the anionic biopolymer is obtained and/or derived from an animal such as a mammal (e.g., a bovine, goat, sheep, or human).

Further exemplary anionic biopolymers that may be present in a complex of the present invention and/or used to prepare a complex of the present invention include, but are not limited to, a gum arabic, high methyl pectin (HMP) (e.g., HMP having an esterification degree of greater than about 50%), kappa-carrageenan, iota-carrageenan, dextran sulfate, sodium hyaluronate, acacia gum, xanthan gum, gellan gum, and/or a plant soluble polysaccharide (e.g., a soy soluble polysaccharide and/or a lupin soluble polysaccharide). In some embodiments, a complex of the present invention and/or used to prepare a complex of the present invention includes a gum arabic, acacia gum, dextran sulfate, sodium hyaluronate, and/or a plant soluble polysaccharide (e.g., a soy soluble polysaccharide and/or a lupin soluble polysaccharide). In some embodiments, a complex of the present invention and/or used to prepare a complex of the present invention includes HMP (e.g., HMP having an esterification degree of greater than about 50%), kappa-carrageenan, iota-carrageenan, xanthan gum, and/or gellan gum. In some embodiments, an anionic biopolymer (e.g., a polysaccharide) present in a complex of the present invention and/or used to prepare a complex of the present invention has a molecular weight (e.g., an average molecular weight) in the range of about 50, 100, or 150 kDa to about 200, 250, or 300 kDa. In some embodiments, an anionic polymer has a molecular weight (e.g., an average molecular weight) of about 50, 100, 150, 200, 250, or 300 kDa.

In some embodiments, a protein, cationic biopolymer, and/or anionic biopolymer present in a complex of the present invention and/or used to prepare a complex of the present invention is/are a food-grade component. A “food-grade component” as used herein refers to a component (e.g., compound, ingredient, biopolymer, etc.) that is safe for consumption by an animal (e.g., a human) and/or intended to be ingested by an animal (e.g., a human). In some embodiments, a protein, cationic biopolymer, and anionic biopolymer of the present invention are each different food-grade components that are present in a complex of the present invention. In some embodiments, a protein, cationic biopolymer, and anionic biopolymer in a complex of the present invention are each obtained and/or derived from a natural product (e.g., a food, plant, animal by-product (e.g., milk), etc.).

In some embodiments, a complex of the present invention comprises lactoferrin, a polysaccharide (e.g., a branched polysaccharide or a linear polysaccharide), and a gelatin. In some embodiments, a complex of the present invention comprises a lactoferrin, a gum arabic, and a gelatin.

A complex of the present invention may be dried, optionally by freeze-drying and/or spraying-drying a composition (e.g., an aqueous composition) comprising the complex. In some embodiments, a dried complex comprises water in an amount of about 0% to about 5% by weight of the dried complex. In some embodiments, a dried complex is devoid of water. In some embodiments, a complex of the present invention is crosslinked, optionally crosslinked using a crosslinker such as, but not limited to, transglutaminase, glyceraldehyde, dialdehydic pectin, and/or genipin.

In some embodiments, a complex of the present invention is a complex coacervate in a liquid (e.g., water and/or a buffer such as phosphate buffered saline). A “complex coacervate” as used herein refers to a liquid droplet that forms by associative liquid-liquid phase separation in mixtures of multivalent, oppositely charged molecules (e.g., oppositely charged biopolymers). A coacervate complex can be dried and/or hardened to form a solid phase. In some embodiments, a complex of the present invention is a multiphase coacervate in a liquid in that the complex coacervate has two or more (e.g., 2, 3, 4, or more) phases. In some embodiments, a complex of the present invention is a multiphase coacervate in a liquid and the complex has two phases (e.g., an internal phase and an outer phase). In some embodiments a complex of the present invention has a coacervate-in-coacervate structure in a liquid and the complex comprises an inner coacervate and an outer coacervate. The inner coacervate may comprise a protein (e.g., lactoferrin) and an anionic biopolymer (e.g., a polysaccharide) and/or the outer coacervate may comprise an anionic biopolymer (e.g., a polysaccharide) and a cationic biopolymer (e.g., a gelatin). In some embodiments, a complex of the present invention is not a binary coacervate complex, which is a complex coacervate that is formed by only two different molecules (e.g., two different biopolymers). In some embodiments, a complex of the present invention is an interpolymeric complex. An “interpolymeric complex” as used herein refers to a co-precipitate or aggregate comprising a protein, cationic biopolymer, and anionic biopolymer that is formed via electrostatic interactions. In some embodiments, an anionic biopolymer and/or cationic biopolymer encapsulate a protein in a complex of the present invention.

A complex of the present invention may be a particle. In some embodiments, the complex is a nanoparticle. In some embodiments, the complex is a microparticle. A complex of the present invention may have a size (e.g., a diameter) in at least one dimension of about 50, 75, 100, or 125 nm to about 150, 200, 250, 300, 350, 400, 450, 500, 550, 600, 650, 700, 750, 800, 850, 900, 1000, 2000, 3000, 4000, 5000, or 6000 nm or more, optionally as measured using microscopy (e.g., optical microscopy, confocal microscopy, scanning electron microscopy (SEM) and/or transmission electron microscopy (TEM)) and/or dynamic light scattering (DLS). In some embodiments, the particle has a size (e.g., a diameter) in at least one dimension of about 25, 50, 75, 100, 125, 150, 200, 250, 300, 350, 400, 450, 500, 550, 600, 650, 700, 750, 800, 850, 900, 1000, 2000, 3000, 4000, 5000, or 6000 nm or more. In some embodiments, a complex of the present invention in a liquid composition (e.g., an aqueous composition) has an average size (e.g., diameter) of about 50 or 100 nm to about 150, 200, 300, 400, 500, 600, 700, 800, 900, 1000, 2000, 3000, 4000, 5000, or 6000 nm or more. In some embodiments, a plurality of complexes of the present invention, complexes prepared according to a method of the present invention, and/or complexes present in a composition of the present invention have a Dv(50) of about 25, 50, 75, 100, 125, 150, 200, 250, 300, 350, 400, 450, 500, 550, 600, 650, 700, 750, 800, 850, 900, 1000, 2000, 3000, 4000, 5000, or 6000 nm or more, optionally as measured using microscopy (e.g., SEM and/or TEM) and/or DLS.

In some embodiments, a complex of the present invention comprises an active ingredient. The active ingredient may be present within (e.g., entrapped and/or encapsulated within) the complex. In some embodiments, the active ingredient may be bound (e.g., covalently and/or noncovalently) to a protein, anionic biopolymer, and/or cationic biopolymer present in the complex. Exemplary active ingredients include, but are not limited to, amino acids (e.g., tryptophan, leucine, phenylalanine, cysteine, and/or tyrosine), vitamin E, iron, vitamin A, vitamin D, and any combination thereof.

A complex of the present invention may have improved (e.g., increased) storage, stability (e.g., thermal stability), activity (e.g., antiviral and/or antibacterial activity), and/or function for a biopolymer (e.g., a protein, cationic biopolymer, and/or anionic biopolymer) present in the complex compared to the storage, stability, activity, and/or function of the biopolymer alone (i.e., the biopolymer not present in a complex of the present invention). In some embodiments, a complex of the present invention provides increased stability for a biopolymer (e.g., a protein) present in the complex compared to the stability of the biopolymer alone.

In some embodiments, upon storage at about 4° C. to about 25° C. in a closed container for about 1, 2, 3, 4, 5, 6, 7, 8, 9, 10, 11, or 12 month(s), the solubility of a complex of the present invention in a composition (e.g., water, a buffer, a milk (e.g., skim milk), and/or an acid whey beverage) remains within about 30% of its original solubility prior to storage (e.g., the solubility of the complex in the composition at initial formation of the complex and/or day 1 of storage). In some embodiments, upon storage at about 4° C. to about 25° C. in a closed container for about 1, 2, 3, 4, 5, 6, 7, 8, 9, 10, 11, or 12 month(s), the amount of a biopolymer (e.g., a protein, cationic biopolymer, and/or anionic biopolymer) present in a complex of the present invention, optionally in a composition (e.g., water, a buffer, a milk (e.g., skim milk), and/or an acid whey beverage), remains within about 30% as compared to the amount of the biopolymer present in the complex prior to storage (e.g., the amount of the biopolymer present in the complex at initial formation of the complex and/or day 1 of storage), optionally as measured by chromatography (e.g., high-performance liquid chromatography), an assay (e.g., ELISA), and/or spectroscopy (e.g., circular dichroism and/or UV-vis). In some embodiments, an activity (e.g., bioactivity, antiviral activity, and/or antibacterial activity) and/or function of a biopolymer (e.g., a protein, cationic biopolymer, and/or anionic biopolymer) present in a complex of the present invention, upon storage at about 4° C. to about 25° C. in a closed container for about 1, 2, 3, 4, 5, 6, 7, 8, 9, 10, 11, or 12 month(s) and optionally in a composition (e.g., water, a buffer, a milk (e.g., skim milk), and/or an acid whey beverage), remains within about 30% of the activity (e.g., bioactivity) and/or function of the biopolymer present in the complex prior to storage (e.g., the activity and/or function of the biopolymer present in the complex at initial formation of the complex and/or day 1 of storage). In some embodiments, a physiochemical property (e.g., turbidity and/or particle size) of a complex of the present invention, upon storage at about 4° C. to about 25° C. in a closed container for about 1, 2, 3, 4, 5, 6, 7, 8, 9, 10, 11, or 12 month(s) and optionally in a composition (e.g., water, a buffer, a milk (e.g., skim milk), and/or an acid whey beverage), remains within about 30% of its original physiochemical property of the complex prior to storage (e.g., the physiochemical property of the complex at initial formation of the complex and/or day 1 of storage).

In some embodiments, the antimicrobial capacity and/or activity (e.g., the antibacterial activity on Gram-positive and/or Gram-negative bacteria and/or the antiviral activity) of a biopolymer (e.g., a protein, cationic biopolymer, and/or anionic biopolymer) present in a complex of the present invention, upon storage at about 4° C. to about 25° C. in a closed container for about 1, 2, 3, 4, 5, 6, 7, 8, 9, 10, 11, or 12 month(s) and optionally in a composition (e.g., water, a buffer, a milk (e.g., skim milk), and/or an acid whey beverage), is retained and/or within about 30% of the antimicrobial capacity and/or activity of the biopolymer prior to storage (e.g., the antibacterial and/or antiviral activity of the biopolymer present in the complex at initial formation of the complex and/or day 1 of storage).

In some embodiments, a complex of the present invention is present in a composition (e.g., water, a buffer, a milk (e.g., skim milk), and/or an acid whey beverage) that is stored at about 4° C. to about 25° C. in a closed container for about 1, 2, 3, 4, 5, 6, 7, 8, 9, 10, 11, or 12 month(s) and optionally the solubility of the complex in the composition, the retention of a biopolymer (e.g., a protein, cationic biopolymer, and/or anionic biopolymer) in the complex, and/or an activity (e.g., bioactivity, such as antibacterial activity, and/or antiviral activity) and/or function of a biopolymer (e.g., a protein, cationic biopolymer, and/or anionic biopolymer) present in the complex is measured at the end of the storage time period.

In some embodiments, upon storage at about 4° C. to about 25° C. in a closed container for about 1, 2, 3, 4, 5, 6, 7, 8, 9, 10, 11, or 12 month(s) and optionally in a composition (e.g., water, a buffer, a milk (e.g., skim milk), and/or an acid whey beverage), the size (e.g., diameter) in at least one dimension of a complex (e.g., particle) of the present invention remains within ± about 20% of its original size (e.g., the size at initial formation of the complex and/or the size at day 1 of storage). For example, at an initial time point (e.g., the start of day one of the storage time period), the complex (e.g., particle) may have a diameter of about 25 nm to about 6000 nm and after storage at about 4° C. to about 25° C. in a closed container for about 1, 2, 3, 4, 5, 6, 7, 8, 9, 10, 11, or 12 month(s) starting from day one of the storage time period, the complex may have a size that increased or decreased by about 20% or less. In some embodiments, upon storage at about 4° C. to about 25° C. in a closed container for about 1, 2, 3, 4, 5, 6, 7, 8, 9, 10, 11, or 12 month(s), a complex (e.g., particle) of the present invention has a size (e.g., diameter) in at least one dimension that is increased in an amount of less than about 20% compared to its original size. In some embodiments, a dried complex (e.g., particle) of the present invention (e.g., a freeze-dried and/or spray-dried particle and/or a particle that comprises water in an amount of about 0% to about 5% by weight of the dried particle) is stored at about 4° C. to about 25° C. in a closed container for about 1, 2, 3, 4, 5, 6, 7, 8, 9, 10, 11, or 12 month(s) and optionally, at the end of the storage period, the size (e.g., diameter) of the dried complex is measured and/or the dried complex is re-constituted (e.g., dissolved and/or dispersed in) in a composition (e.g., water and/or a buffer) and the size (e.g., diameter) of the complex in the composition is measured. In some embodiments, a complex (e.g., particle) of the present invention is present in a composition (e.g., (water, a buffer, a milk (e.g., skim milk), and/or an acid whey beverage) and is stored at about 4° C. to about 25° C. in a closed container for about 1, 2, 3, 4, 5, 6, 7, 8, 9, 10, 11, or 12 month(s), and optionally the size (e.g., diameter) of the complex in the composition is measured at the end of the storage time period.

In some embodiments, a complex of the present invention provides increased stability for a protein present in the complex after exposure to a temperature in a range of about 70° C. or 75° C. to about 80° C., 85° C., 90° C., 95° C., or 100° C. for about 1, 2, 3, 4, 5, or 10 minute(s) to about 15, 20, 30, 40, 50, or 60 minutes compared to the stability of the protein alone (i.e., the protein not present in a complex of the present invention) after exposure to the same conditions (e.g., same temperature for the same period of time). In some embodiments, the complex may be present in a composition (e.g., water, a buffer, a milk (e.g., skim milk), and/or an acid whey beverage) and exposed to the temperature. In some embodiments, increased stability for the protein is determined and/or demonstrated by reduced degradation of the protein in the complex compared to the degradation of the protein alone. In some embodiments, after exposure to a temperature in a range of about 70° C. or 75° C. to about 80° C., 85° C., 90° C., 95° C., or 100° C. for about 1, 2, 3, 4, 5, or 10 minute(s) to about 15, 20, 30, 40, 50, or 60 minutes, a protein present in a complex of the present invention is degraded by less than about 30% such as about 25%, 20%, 15%, 10%, 5%, 1%, or less, optionally as measured by chromatography (e.g., high-performance liquid chromatography), an assay (e.g., ELISA), and/or spectroscopy (e.g., circular dichroism). In some embodiments, after exposure to a temperature in a range of about 100° C., 105° C., 110° C., 115° C., or 120° C. to about 125° C., 130° C., 135° C., 140° C., or 145° C. for about 2, 5, 10, 20, or 30 seconds to about 40, 50, or 60 seconds (e.g., a high-temperature short time (HTST) or an ultra-high temperature (UHT) treatment at about 145° C. for about 2 seconds to about 60 seconds), a protein present in a complex of the present invention is degraded by less than about 30% such as about 25%, 20%, 15%, 10%, 5%, 1% or less, optionally as measured by chromatography (e.g., high-performance liquid chromatography), an assay (e.g., ELISA), and/or spectroscopy (e.g., circular dichroism). In some embodiments, after exposure to a temperature in a range of about 100° C., 105° C., 110° C., 115° C., or 120° C. to about 125° C., 130° C., 135° C., 140° C., or 145° C. for about 2, 5, 10, 20, or 30 seconds to about 40, 50, or 60 seconds (e.g., a high-temperature short time (HTST) or an ultra-high temperature (UHT) treatment at about 145° C. for about 2 seconds to about 60 seconds), a protein present in a complex of the present invention is degraded by less than about 20% such as about 15%, 10%, 5%, 1% or less, optionally as measured by chromatography (e.g., high-performance liquid chromatography), an assay (e.g., ELISA), and/or spectroscopy (e.g., circular dichroism).

In some embodiments, the antimicrobial capacity and/or activity (e.g., the antibacterial activity on Gram-positive and/or Gram-negative bacteria and/or the antiviral activity) of a protein present in a complex of the present invention, after exposure to a temperature in a range of about 70° C. or 75° C. to about 80° C., 85° C., or 90° C. for about 30 seconds to about 2 minutes, is retained and/or improved (e.g., increased) as compared to the antimicrobial capacity and/or activity of the protein alone, optionally after exposure to the same conditions (e.g., same temperature and time). In some embodiments, the complex may be present in a composition (e.g., water, a buffer, a milk (e.g., skim milk), and/or an acid whey beverage) and exposed to the temperature. In some embodiments, the antimicrobial capacity and/or activity (e.g., the antibacterial activity on Gram-positive and/or Gram-negative bacteria and/or the antiviral activity) of a protein present in a complex of the present invention, after exposure to a temperature in a range of about 70° C. or 75° C. to about 80° C., 85° C., or 90° C. for about 30 seconds to about 2 minutes, is retained and/or increased as compared to the antimicrobial capacity and/or activity of the protein alone, optionally after exposure to the same conditions (e.g., same temperature and time).

In some embodiments, upon exposure to a temperature in a range of about 70° C. or 75° C. to about 80° C., 85° C., or 90° C. for about 30 seconds to about 2 minutes or to a temperature in a range of about 100° C., 105° C., 110° C., 115° C., or 120° C. to about 125° C., 130° C., 135° C., 140° C., or 145° C. for about 2, 5, 10, 20, or 30 seconds to about 40, 50, or 60 seconds, the solubility of a complex of the present invention in a composition (e.g., water, a buffer, a milk (e.g., skim milk), and/or an acid whey beverage) remains within about 30% of its original solubility prior to the exposure (e.g., the solubility of the complex in the composition at initial formation of the complex and/or immediately prior to the exposure). In some embodiments, upon exposure to a temperature in a range of about 70° C. or 75° C. to about 80° C., 85° C., or 90° C. for about 30seconds to about 2 minutes or to a temperature in a range of about 100° C., 105° C., 110° C., 115° C., or 120° C. to about 125° C., 130° C., 135° C., 140° C., or 145° C. for about 2, 5, 10, 20, or 30 seconds to about 40, 50, or 60 seconds, the amount of a biopolymer (e.g., a protein, cationic biopolymer, and/or anionic biopolymer) present in a complex of the present invention that is in a composition (e.g., water, a buffer, a milk (e.g., skim milk), and/or an acid whey beverage) remains within about 30% as compared to the amount of the biopolymer present in the complex prior to the exposure (e.g., the amount of the biopolymer present in the complex at initial formation of the complex and/or immediately prior to the exposure), optionally as measured by chromatography (e.g., high-performance liquid chromatography), an assay (e.g., ELISA), and/or spectroscopy (e.g., circular dichroism and/or UV-vis). In some embodiments, an activity (e.g., bioactivity, antiviral activity, and/or antibacterial activity) and/or function of a biopolymer (e.g., a protein, cationic biopolymer, and/or anionic biopolymer) present in a complex of the present invention that is in a composition (e.g., water, a buffer, a milk (e.g., skim milk), and/or an acid whey beverage), upon exposure of the composition to a temperature in a range of about 70° C. or 75° C. to about 80° C., 85° C., or 90° C. for about 30 seconds to about 2 minutes or to a temperature in a range of about 100° C., 105° C., 110° C., 115° C., or 120° C. to about 125° C., 130° C., 135° C., 140° C., or 145° C. for about 2, 5, 10, 20, or 30 seconds to about 40, 50, or 60 seconds, remains within about 30% of the activity (e.g., bioactivity) and/or function of the biopolymer present in the complex prior to the exposure (e.g., the activity and/or function of the biopolymer present in the complex at initial formation of the complex and/or immediately prior to the exposure). In some embodiments, a physiochemical property (e.g., turbidity and/or particle size) of a complex of the present invention that is present in in a composition (e.g., water, a buffer, a milk (e.g., skim milk), and/or an acid whey beverage), upon exposure to a temperature in a range of about 70° C. or 75° C. to about 80° C., 85° C., or 90° C. for about 30 seconds to about 2 minutes or to a temperature in a range of about 100° C., 105° C., 110° C., 115° C., or 120° C. to about 125° C., 130° C., 135° C., 140° C., or 145° C. for about 2, 5, 10, 20, or 30 seconds to about 40, 50, or 60 seconds, remains within about 30% of its original physiochemical property of the complex prior to the exposure (e.g., the physiochemical property of the complex at initial formation of the complex and/or immediately prior to the exposure).

In some embodiments, the antimicrobial capacity and/or activity (e.g., the antibacterial activity on Gram-positive and/or Gram-negative bacteria and/or the antiviral activity) of a biopolymer (e.g., a protein, cationic biopolymer, and/or anionic biopolymer) present in a complex of the present invention that is present in a composition (e.g., water, a buffer, a milk (e.g., skim milk), and/or an acid whey beverage), upon exposure to a temperature in a range of about 70° C. or 75° C. to about 80° C., 85° C., or 90° C. for about 30 seconds to about 2 minutes or to a temperature in a range of about 100° C., 105° C., 110° C., 115° C., or 120° C. to about 125° C., 130° C., 135° C., 140° C., or 145° C. for about 2, 5, 10, 20, or 30 seconds to about 40, 50, or 60 seconds, is retained and/or within about 30% of the antimicrobial capacity and/or activity of the biopolymer prior to the exposure (e.g., the antibacterial and/or antiviral activity of the biopolymer present in the complex at initial formation of the complex and/or immediately prior to the exposure).

In some embodiments, a complex of the present invention increases the thermal stability of a biopolymer (e.g., a protein, cationic biopolymer, and/or anionic biopolymer) present in the complex compared to the thermal stability of the biopolymer alone. For example, the presence of the biopolymer in the complex may reduce or avoid denaturation (e.g., thermal denaturation such as thermal denaturation during the preparation of a food product comprising the biopolymer) of the biopolymer compared to the amount of denaturation of the biopolymer alone (i.e., the biopolymer not present in a complex of the present invention) under the same conditions. In some embodiments, a complex of the present invention increases the thermal stability of lactoferrin present in the complex upon exposure to a temperature for a period of time (e.g., a temperature in a range of about 70° C. or 75° C. to about 80° C., 85° C., 90° C., 95° C., or 100° C. for about 1, 2, 3, 4, 5, or 10 minute(s) to about 15, 20, 30, 40, 50, or 60 minutes) compared to the thermal stability of lactoferrin alone upon exposure to the same conditions (e.g., same temperature and time). In some embodiments, a complex of the present invention increases the stability (e.g., thermal stability) structure, activity, and/or function of a biopolymer (e.g., lactoferrin) present in the complex upon exposure to a pH in a range of about 6.5 to about 7.5 compared to the stability, structure, activity, and/or function of the biopolymer alone upon exposure to the same conditions (e.g., the same pH).

Activity and/or function of a biopolymer (e.g., a protein, cationic biopolymer, and/or anionic biopolymer) present in a complex of the present invention may be increased compared to the activity and/or function of the biopolymer alone. For example, after storing a complex of the present invention for a period of time (e.g., storing a dried complex in a closed container at a temperature in a range of about 20° C. to about 30° C. for about 1, 2, 3, 4, 5, or 6 months) and/or heating a complex (e.g., at a temperature in a range of about 70° C. or 75° C. to about 80° C., 85° C., 90° C., 95° C., or 100° C. for about 1, 2, 3, 4, 5, or 10 minute(s) to about 15, 20, 30, 40, 50, or 60 minutes), a biopolymer (e.g., protein) present in the complex may have an activity and/or function that is increased compared to the activity and/or function of the biopolymer alone after the same storage and/or heating conditions. For example, a complex comprising lactoferrin, an anionic biopolymer, and a cationic biopolymer may provide an increased activity and/or function (e.g., increased antimicrobial activity) after storing and/or heating the complex compared to the activity and/or function of lactoferrin after the same storage and/or heating conditions. In some embodiments, other than an anionic biopolymer and/or cationic biopolymer, a complex of the present invention is devoid of an agent configured to preserve and/or stabilize the activity, function, and/or stability (e.g., thermal stability) of a protein (e.g., lactoferrin) present in the complex.

In some embodiments, a complex of the present invention may provide increased bioavailability for a biopolymer (e.g., a protein, cationic biopolymer, and/or anionic biopolymer) present in a complex of the present invention compared to the bioavailability of the biopolymer alone. Bioavailability may be determined following administration of the complex to a subject, optionally wherein administration comprises ingestion of the complex by the subject. In some embodiments, a biopolymer present in a complex of the present invention has increased bioavailability in the intestinal tract of a subject compared to the bioavailability of the biopolymer alone. In some embodiments, a biopolymer present in a complex of the present invention has reduced enzymatic hydrolysis (e.g., reduced enzymatic hydrolysis in the gastric phase of digestion in a subject) compared to the amount of enzymatic hydrolysis of the biopolymer alone.

According to some embodiments, a composition comprising a complex of the present invention is provided and/or an article comprising a complex of the present invention is provided. In some embodiments, the composition and/or article comprises a plurality of complexes of the present invention. In some embodiments, the composition and/or article comprises a complex of the present invention and a carrier. The carrier may be a liquid such as, but not limited to, water and/or an oil. In some embodiments, a composition of the present invention is an aqueous composition. In some embodiments, a composition of the present invention is a suspension, optionally wherein a complex of the present invention is suspended in the composition. In some embodiments, a complex of the present invention stabilizes a composition comprising the complex, optionally wherein the composition is an emulsion. In some embodiments, the carrier is a solid (e.g., a particulate and/or powder) and a plurality of complexes of the present invention may be present together with the solid, optionally present on, below, combined with and/or mixed with the solid. In some embodiments, the carrier is a food-grade component such as, but not limited to, a milk, a dairy beverage, an infant formula, and/or an instant beverage powder. One or more excipient(s) such as, but not limited to, gum arabic, sodium caseinate, maltodextrin may be present in a composition of the present invention.

In some embodiments, a composition and/or article of the present invention is a food product, nutritional supplement, therapeutic drink, and/or cosmetic. In some embodiments, a complex of the present invention may be present in a food product. In some embodiments, the food product is a dairy product (e.g., milk, yogurt, etc.). In some embodiments, the composition is an infant formula and/or a nutritional supplement (optionally a drink).

In some embodiments, upon storage at about 4° C. to about 25° C. in a closed container for about 1, 2, 3, 4, 5, 6, 7, 8, 9, 10, 11, or 12 month(s), the turbidity of a composition of the present invention remains within about 30% of the original turbidity of the composition prior to storage (e.g., the turbidity at initial formation of the composition and/or day 1 of storage), optionally as measured by spectroscopy such as ultraviolet-visible (UV-vis) spectroscopy. In some embodiments, following storage at about 4° C. to about 25° C. in a closed container for about 1, 2, 3, 4, 5, 6, 7, 8, 9, 10, 11, or 12 month(s), a composition of the present invention has no undesirable sensory property or minimal undesirable sensory property (e.g., undesirable taste such as a sour and/or bitter taste, an undesirable color, undesirable appearance, and/or an undesirable texture).

Provided according to some embodiments of the present invention is a method for preparing a complex of the present invention. In some embodiments, the method comprises providing a composition comprising a protein, an anionic biopolymer, and a cationic biopolymer at a pH in a range of about 3, 3.5, or 4 to about 4.5 or 5; and mixing the composition, thereby providing the complex. The composition comprising the protein, anionic biopolymer, and cationic biopolymer may be an aqueous composition that optionally includes a buffer. In some embodiments, the composition used to prepare a complex of the present invention has a pH of about 3, 3.5, 4, 4.5, or 5. In some embodiments, the composition used to prepare a complex of the present invention comprises a salt, optionally wherein the composition comprises a salt in an amount of about 0.1, 0.5, 1, or 5 mM to about 10, 15, 20, 25, or 30 mM. In some embodiments, the composition used to prepare a complex of the present invention comprises a salt in an amount of about 0.1, 0.5, 1, 5, 10, 15, 20, 25, or 30 mM.

A composition used to prepare a complex of the present invention may comprise a protein, an anionic biopolymer, and/or a cationic biopolymer each independently in an amount of about 0.01%, 0.1%, 0.5%, 1%, 2%, 3%, 4%, or 5% to about 6%, 7%, 8%, 9%, or 10% by weight of the composition. In some embodiments, a composition used to prepare a complex of the present invention comprises a protein, an anionic biopolymer, and/or a cationic biopolymer each independently in an amount of about 0.01%, 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% by weight of the composition. In some embodiments, a composition used to prepare a complex of the present invention may comprise a protein, an anionic biopolymer, and/or a cationic biopolymer each independently in an amount of about 0.01%, 0.1%, 0.5%, 1%, 2%, or 3% by weight of the composition. In some embodiments, a composition used to prepare a complex of the present invention comprises an anionic biopolymer and a protein in a weight ratio of about 0.5:1 to about 1:5 (anionic biopolymer:protein) such as in a weight ratio of about 0.5:1, 1:1, 1:2, 1:3, 1:4, or 1:5 (anionic biopolymer:protein). In some embodiments, a composition used to prepare a complex of the present invention comprises a protein and a cationic biopolymer in a weight ratio of about 1:1 to about 10:1 (protein:cationic biopolymer) such as in a weight ratio of about 1:1, 1:2, 1:3, 1:4, 1:5, 1:6, 1:7, 1:8, 1:9, or 1:10 (protein:cationic biopolymer). In some embodiments, a composition used to prepare a complex of the present invention comprises an anionic biopolymer, protein, and cationic biopolymer in a weight ratio of about 1:3:1, about 4:5:1, or about 3:6: 1 (anionic biopolymer:protein:cationic biopolymer). In some embodiments, a composition used to prepare a complex of the present invention has a total concentration of a protein, anionic biopolymer, and cationic biopolymer in the composition in an amount of about 10% by weight of the composition or less such as about 0.05%, 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% by weight of the composition. In some embodiments, a composition used to prepare a complex of the present invention has a total concentration of a protein, anionic biopolymer, and cationic biopolymer in the composition in an amount of about 5% by weight of the composition or less such as about 0.05%, 0.1%, 0.5%, 1%, 1.5%, 2%, 2.5%, 3%, 3.5%, 4%, 4.5%, or 5% by weight of the composition.

Mixing a composition used to prepare a complex of the present invention may be carried out using methods known in the art. In some embodiments, mixing a composition used to prepare a complex of the present comprises mixing the composition for about 15, 20, 25, or 30 minutes to about 35, 40, 45, 50, 55, or 60 minutes, optionally at a temperature in a range of about 20° C., 25° C., or 30° C. to about 35° C., 40° C., 45° C., 50° C., 55° C., or 60° C.

In some embodiments, a method of the present invention comprises forming an intermediate composition that includes a protein and an anionic biopolymer, optionally wherein the intermediate composition is an aqueous composition. In some embodiments, a method of the present invention comprises combining an anionic biopolymer and a protein to provide an intermediate composition (optionally an aqueous composition) and mixing the intermediate composition to optionally form an intermediate complex comprising the anionic biopolymer and the protein. In some embodiments, mixing the intermediate composition may be carried out for about 15, 20, 25, or 30 minutes to about 35, 40, 45, 50, 55, or 60 minutes, optionally at a temperature in a range of about 20° C., 25° C., or 30° C. to about 35° C., 40° C., 45° C., 50° C., 55° C., or 60° C. The method may further comprise adding a cationic biopolymer to the intermediate composition to provide a composition comprising the anionic biopolymer and protein (optionally in the form of an intermediate complex) and further comprising the cationic biopolymer and mixing the composition to thereby form the complex.

A method of the present invention may further comprise hardening a complex of the present invention. In some embodiments, hardening the complex comprises adjusting the temperature of a composition comprising a complex of the present invention to a temperature in a range of about 5° C. to about 10° C. and exposing the composition to a temperature in a range of about 5° C. to about 10° C. for about 1 or 2 hour(s) to about 3, 4, 5, or 6 hours.

In some embodiments, a method of the present invention comprises isolating and/or obtaining a complex of the present invention from a composition. Isolating and/or obtaining a complex of the present invention from a composition may comprise centrifuging, drying, freeze-drying, filtering. and/or spray-drying the composition to thereby isolate and/or obtain the complex. In some embodiments, the isolated and/or obtained complex is a dried complex, optionally wherein the dried complex comprises water in an amount of about 0% to about 5% by weight of the dried complex. A dried complex may be in the form of a particulate and/or powder. In some embodiments, the isolated and/or obtained complex is milled, ground, and/or micronized to provide a desired size such as particles having a size (e.g., diameter) of less than about 1 mm. In some embodiments, a complex of the present invention may be crosslinked using a crosslinker such as, but not limited to, transglutaminase, glyceraldehyde, dialdehydic pectin, and/or genipin. A protein, cationic biopolymer, and/or anionic biopolymer may be crosslinked in a complex of the present invention.

In some embodiments, a method of the present invention comprises combining a complex of the present invention with a carrier, optionally wherein the carrier is a liquid or a solid. In some embodiments, a complex of the present invention is added to a food-grade component and/or to a food product (e.g., a beverage or a powder formula). Combining a complex of the present invention to a carrier may comprise mixing an isolated and/or obtained complex into a carrier and/or mixing a complex of the present invention that is present in a composition (e.g., an aqueous composition) into a carrier. In some embodiments, a complex of the present invention is dispersed in a carrier optionally by mixing, stirring, homogenizing, and/or the like at a temperature in a range of about 20° C., 25° C., or 30° C. to about 35° C., 40° C., 45° C., 50° C., 55° C., or 60° C.

A method of the present invention may comprise providing a therapeutic effect and/or benefit to a subject and/or treating and/or preventing a disease, disorder, and/or condition in a subject. The method may comprise administering (e.g., orally administering) a complex of the present invention and/or a composition of the present invention to a subject, optionally wherein the administering comprises the subject ingesting the complex and/or composition.

In some embodiments, a method of the present invention comprises administering a therapeutically effective amount of a complex of the present invention and/or a composition of the present invention to a subject. As used herein, the term “therapeutically effective amount” refers to an amount of complex and/or composition of the present invention that elicits a therapeutically useful response in a subject. Those skilled in the art will appreciate that the therapeutic effects need not be complete or curative, as long as some benefit is provided to the subject.

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom associated with the subject's condition is achieved and/or there is a delay in the progression of the symptom. In some embodiments, the severity of a symptom associated with iron deficiency may be reduced in a subject compared to the severity of the symptom in the absence of a method of the present invention. In some embodiments, a complex of the present invention and/or a composition of the present invention is administered to a subject to improve iron delivery and/or adsorption in a subject and/or to treat a disease and/or a symptom thereof.

In some embodiments, a complex of the present invention and/or a composition of the present invention may be administered in a treatment effective amount. A “treatment effective” amount as used herein is an amount that is sufficient to treat (as defined herein) a subject. Those skilled in the art will appreciate that the therapeutic effects need not be complete or curative, as long as some benefit is provided to the subject. In some embodiments, a treatment effective amount may be achieved by administering a complex and/or composition of the present invention to a subject, optionally wherein the administering comprises the subject ingesting the complex and/or composition.

The terms “prevent,” “preventing” and “prevention” (and grammatical variations thereof) refer to avoidance, reduction and/or delay of the onset of a symptom associated with a disease, disorder, or condition and/or a reduction in the severity of the onset of symptom associated with a disease, disorder, or condition relative to what would occur in the absence of a method of the present invention. The prevention can be complete, e.g., the total absence of the symptom. The prevention can also be partial, such that the occurrence of the symptom in the subject and/or the severity of onset is less than what would occur in the absence of a method of the present invention. In some embodiments, a complex of the present invention and/or a composition of the present invention is administered to a subject to prevent a disease, disorder, or condition.

In some embodiments, a complex of the present invention and/or a composition of the present invention may be administered in a prevention effective amount. A “prevention effective” amount as used herein is an amount that is sufficient to prevent (as defined herein) a symptom associated with a disease, disorder, or condition in a subject. Those skilled in the art will appreciate that the level of prevention need not be complete, as long as some benefit is provided to the subject. In some embodiments, a prevention effective amount may be achieved by administering a complex and/or composition of the present invention to a subject, optionally wherein the administering comprises the subject ingesting the complex and/or composition.

The present invention finds use in both veterinary and medical applications. Subjects suitable to be treated with a method of the present invention include, but are not limited to, mammalian subjects. Mammals of the present invention include, but are not limited to, canines, felines, bovines, caprines, equines, ovines, porcines, rodents (e.g. rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Human subjects of both genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments of the present invention, the subject is a human adolescent and/or adult.

A method of the present invention may also be carried out on animal subjects, particularly mammalian subjects such as mice, rats, dogs, cats, livestock and horses for veterinary purposes, and/or for drug screening and drug development purposes.

In some embodiments, the subject is “in need of” or “in need thereof” a method of the present invention, for example, the subject has findings typically associated with a disease, disorder, or condition, is suspected to have a disease, disorder, or condition, and/or the subject has a disease, disorder, or condition.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

Effective delivery of the bioactive protein, lactoferrin (LF), remains a challenge as it is sensitive to environmental changes and easily denatured during heating, restricting its application in functional food products. To overcome these challenges, polyelectrolyte ternary complexes of LF with gelatin (G) and negatively charged polysaccharides were formulated. Linear, highly charged polysaccharides were able to form interpolymeric complexes with LF and G, while coacervates were formed with branched polysaccharides. A unique multiphase coacervate was observed in the gum Arabic GA-LF-G complex, where a special coacervate-in-coacervate structure was found. The ternary complexes made with GA, soy soluble polysaccharide (SSP), or high methoxyl pectin (HMP) preserved the protein structures and demonstrated enhanced thermal stability of LF. The GA-LF-G complex was especially stable with >90% retention of the native LF after being treated at 90° C. for 2 min in a water bath or at 145° C. for 30 s, while the LF control had only ˜7% undenatured LF under both conditions. In comparison to untreated LF, LF in ternary complex retained significant antibacterial activity on both Gram-positive and Gram-negative bacteria, even after heat treatment. These ternary complexes of LF maintain the desired functionality of LF, thermal stability and antibacterial activity, in the final products. The ternary complex structure, particularly the multiphase coacervate, may serve as a template for the encapsulation and stabilization of other bioactives and peptides.

### 1. Introduction

Reported here is the formulation of ternary complexes of positively charged LF and G with five different negatively charged polysaccharides to determine if the complexes can improve the structural stability and/or antibacterial activity of LF during thermal processing. As described below, the complexes formed by polysaccharides with different charge density and chain flexibility demonstrated different structures and thermal stability. The physicochemical properties of these ternary complexes were investigated using turbidity, zeta-potential and particle size, and microscopic analysis. The structural changes and retention ratios of LF after thermal treatment were then evaluated through intrinsic fluorescence, circular dichroism (CD) spectroscopy, SDS-PAGE, and HPLC.

### 2. Materials and Methods

### 2.1 Materials

Bovine Lactoferrin (LF) (Bioferrin 2000, Iron>15 mg/100 g) was obtained from Glanbia Nationals, Inc. (Fitchburg, WI, USA). Soy soluble polysaccharide (SSP) was provided by Fuji Oil (Izumisano-shi, Japan). High methoxyl pectin (HMP), Kappa carrageenan (Kappa), and Iota carrageenan (Iota) were provided by Tic gum (Riverside, MD, USA). Gum Arabic (GA) was provided by Colony Gum (Monroe, NC, USA). Gelatin (Knox unflavored gelatin) was purchased from the local grocery store (Target, Ithaca, NY, USA). Fluorescein isothiocyanate isomer I (FITC) was purchased from Sigma-Aldrich (St. Louis, MO, USA). Trifluoroacetic acid, acetonitrile (HPLC grade), hydrochloric acid, sodium hydroxide, and dimethyl sulfoxide (DSMO) were purchased from Fisher Scientific (Hampton, NH, USA). The reagents for SDS-PAGE were purchased from Bio-Rad Laboratories (Hercules, CA, USA). Bicinchoninic acid (BCA) Assay Kit II was purchased from BioVision (Waltham, MA, USA). Coomassie Brilliant Bule G-250 was purchased from bioWORLD (Dublin, OH, USA). Luria broth (LB) and LB agar were purchased from Sigma-Aldrich (St. Louis, MO, USA). Other chemicals and reagents were purchased from Fisher Scientific (Hampton, NH, USA).

### 2.2 Preparation of Ternary Complex

LF, SSP, and GA solutions were prepared at 1% or 0.2% (w/v) by dissolving the biopolymers into Milli-Q water and mixed for 2 hours at room temperature (25° C.). HMP, Kappa, and Iota solutions were prepared at 0.2% (w/v) by dissolving the biopolymers into Milli-Q water and heated up to at least 60° C., and mixed for 2 h. Gelatin (G) solutions were prepared at 1% or 0.2% (w/v) by dissolving the biopolymers into Milli-Q water and mixed for 2 h at 45° C. All solutions were cooled to 4° C. allowed to settle overnight to ensure full hydration.

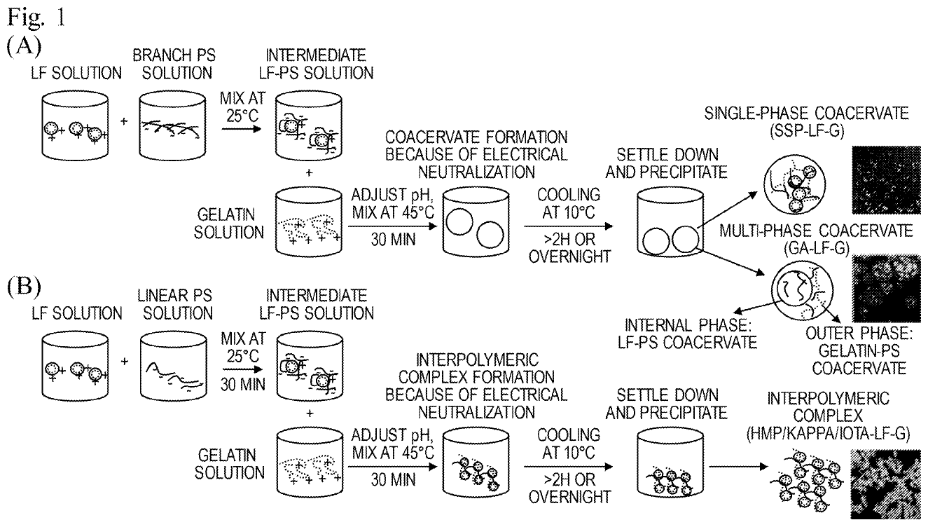

FIG. 1 shows an exemplary schematic for the ternary complex formation procedure using branched polysaccharides (FIG. 1, panel a) and linear polysaccharides (FIG. 1, panel b). The ternary complex was prepared at a total concentration of 1% or 0.2% (w/v) by proportionally adding biopolymer solutions at the same concentration. The proportion of biopolymers added was designed according to the ternary plot with a ratio interval of 10% (mass ratio of total biopolymers). All the biopolymer solutions were adjusted to target pH at pH 4 or pH 7 first. These pH levels were selected to represent the pH when LF has a high positive charge (pH 4) and a low positive charge (pH 7). Then, different polysaccharide solutions were mixed with LF solutions first for 30 minutes at room temperature (25° C.) to form an intermediate complex. The mixtures were then heated up to 45° C. and mixed with preheated gelatin solutions at 45° C. for another 30 min. After fully mixing of all the biopolymers, the solutions were hardened under 10° C. for 2 h. To isolate the complex, the ternary mixtures were centrifuged at 10,000g for 25 min at 4° C. The pellets were collected and frozen (−20° C.) overnight and then freeze dried using the freeze-dryer (Labcono, Kansas, MO, USA) for 48-36 h, at a vacuum pressure of −0.175 mBar and moisture collector temperature of −53° C. The supernatant was collected and stored at 4° C. for complexation efficiency measurements.

### 2.3 Characterization of Complex

### 2.3.1 Turbidity Measurements

The turbidity of ternary mixtures was measured using a UV-Vis light spectrophotometer (UV-2600, SHIMADZU Co., Japan). The transmittance was measured at 600 nm in 1 cm path-length quartz cuvettes at room temperature. Milli-Q water was used as blank (100% transmittance). The turbidity (T) was calculated according to the following equation (Eq.1):

$$T = - ln \frac{I}{{I}_{0}} \qquad ( Eq . 1 )$$

where I is the transmittance intensity of samples and I₀ is the transmittance intensity of blank.

### 2.3.2 Particle Size Measurements

The average diameter and particle size distribution of ternary mixtures were analyzed using the dynamic light scattering instrument (Zetasizer Nano-ZS, Malvern, Germany). All analyses were performed at 25° C. in 1-cm path-length cuvettes at the wavelength of 633 nm and a backscattering angle of 173°. The refractive index of dispersant was set as 1.33 and the refractive index of material was set as 1.45. Analysis was done in triplicate with at least 11 runs for each measurement.

### 2.3.3 Zeta-Potential Measurements

The zeta-potential of ternary mixtures was measured using the Nano-ZS (Malvern, Germany) using Smouluchwski mode. The software could determine the suitable type of measurements after obtaining the sample conductivity using the voltage of about 150 V. Samples were measured in triplicate with 10 runs for each measurement.

### 2.3.5 Morphology Characterization

### 2.3.5.1. Confocal Microscopy Analysis

LF was labeled with FITC (Fluorescein isothiocyanate) for imaging purposes using the following procedure. LF was dissolved in 10 mM carbonate buffer (pH 10.0) at 5 w/v %. FITC was dissolved in DMSO at 10 w/v %. The LF solution was mixed with FITC at a volume ratio of 20:1 for 3 to 4 h at room temperature in the dark. The mixture was washed through a gel filtration column (filled with Sephadex G-25) to remove the excessive FITC. The green-yellow solution was collected from the column and confirmed with the labeled protein by UV-vis spectroscopy. The obtained FTIC-labelled LF solution was freeze-dried for later use in the ternary complexes and confocal microscopy analysis.

The confocal microscopy images of the ternary complex made with FTIC-labelled LF were obtained using the Zeiss LSM 710 confocal laser scanning microscope connected to Inverted Axio Observer.Z1 microscope, using a 40× water immersion objective (NA 1.2). A small aliquot of the complex solutions was transferred to a glass microscope slip and covered with a glass coverslip. Green (confocal) images allowed us to visualize LF in the complexes by using the excitation argon laser (488 nm) and detection light between 500-559 nm. Regular optical images were taken by turning off the laser and turning on the regular white polarized light. The images were analyzed by the instrument software (EZ CS1 version 3.8, Niko, Melville, NY).

### 2.3.5.2. Scanning Electron Microscopy Analysis

The microstructure of the LF ternary complex was visualized using a Field-emission scanning electron microscope (SEM) (Zeiss Gemini 500, Jena, Germany) by the method as described by (Lin et al., 2022). Before taking the microscopy images, fresh ternary mixture samples (˜10 μL) were vacuum-dried overnight and then coated with Au/Pd in a sputter coater (Denton Desk V, NJ, USA).

### 2.3.6 Measurement of Complexation Efficiency and Loading Ratio of LF

The supernatant obtained after centrifuging the ternary mixtures was appropriately diluted and used to quantify the free LF according to the Bradford method (Bradford, 1976). The complexation efficiency was calculated according to the following equation (Eq.2):

$$Complexation efficiency ( C E ) \% = 100 \times \frac{Total L F - Free L F}{Total L F} \qquad ( Eq . 2 )$$

Total LF was the theoretical concentration (w/v %) of LF employed in the ternary mixture; Free LF was the measured concentration (w/v %) of LF in the supernatant.

The freeze-dried complex samples were weighed for yield measurement, which was calculated according to the following equation (Eq. 3):

$$Yield ( \% ) = 100 \times \frac{Mass of freeze - dried complex}{Mass of total solid in biopolymer solutions} \qquad ( Eq . 3 )$$

The loading ratio (mass ratio) of LF in the freeze-dried complex samples was quantified in the redispersed complex samples in PBS buffer pH 7 at a concentration of 0.2 w/v % using the BCA assay. The loading ratio of LF in the freeze-dried complex was calculated according to the following equation (Eq.4):

$$Loading ratio of L F ( \% ) = 100 \times \frac{Concentration of L F in complex solution ( w / v \% )}{Complex concentration ( 0.2 w / v \% )} \qquad ( Eq . 4 )$$

### 2.3.7 Thermal Stability Test of LF in the Ternary Complex

Samples including pure LF and rehydrated ternary complex solutions in water or PBS buffer (10 mM; 0.2 w/v %) at pH7 were loaded into glass tubes (2 mL) and placed into a water bath at different temperatures 75° C. or 90° C. for 2 min and then immersed in an ice-water bath to cool down to ambient temperature (T=25° C.) before further analysis. Samples were also processed at 145° C. using an oil bath and were processed at this temperature for 2 s, 10 s, 30 s, and 60 s. The optical images, turbidity, and particle size of ternary complex solutions before and after thermal treatment was obtained. Intrinsic fluorescence and circular dichroism spectroscopy were analyzed to understand the structural changes of the protein during heating. Electrophoresis analysis and HPLC analysis were used to quantify the LF retention rate after thermal treatment.

### 2.3.8 Electrophoresis Analysis

Unheated and heated LF and ternary complex samples were analyzed using sodium dodecyl sulfate (SDS)-PAGE in a vertical mini gel electrophoresis system (Mini-PRO-TEAN Tetra cell, Bio-Rad, USA). The premixed TGA fast Cast Acrylamide starter kit was used for the preparation of PAGE gels. Twenty microliters of diluted samples (2 mg/mL of protein) were mixed with 2× Laemmli buffer at the ratio of 1:1 and then heated in a boiling water bath for 5 min. Then, 20 μL of mixtures were loaded on the gels for electrophoresis (200 V) for about 30-45 min. The gel was stained in 0.15% (w/v) Coomassie Brilliant R-250 solution which consisted of 50% (v/v) methanol and 10% (v/v) acetic acid for half an hour. Then, the gel was de-stained in de-staining solutions (20% (v/v) methanol and 10% (v/v) acetic acid) for 24 hours.

### 2.3.9 HPLC Analysis

HPLC analysis was developed to quantify the undenatured LF in complex solutions after thermal treatment. All samples were the top liquid solutions avoiding the protein aggregates and filtered through 0.45 μm filter. A reversed-phase HPLC was performed on Agilent 1100/1200 series HPLC systems (Agilent Technologies, CA, USA), equipped with a diode array detector and ChemStation data acquisition program. Detection was carried out at 214 nm. LF separation was performed using the BioZen Intact XB-C8 column (150×4.6 mm, 3.6m; Phenomenex, Torrance CA, USA) at 40° C. A gradient elution was performed using 0.1% trifluoroacetic acid (TFA) in water (A) and 0.1% trifluoroacetic acid in acetonitrile (B) at a mobile phase flow rate of 1.0 mL/min using the following gradient elution: 0-5 min, 5% B; 5-20 min, 5-20% B; 20-25 min, 50-5% B. The injection volume was 10 μL. The concentration of remaining native LF in sample solutions can be measured and quantified according to a standard curve of native LF with a concentration of 0-0.2 w/v % (R²>99%). The LF retention rate, which indicates how much LF remains native after thermal treatment in the complex solution, was calculated using the following equation:

$$L F retention ( \% ) = 100 \times \frac{Concentration of L F after heating}{Concentration of L F before heating} \qquad ( Eq . 5 )$$

### 2.3.10 Circular Dichroism (CD) Spectroscopy Analysis

The secondary structures of lactoferrin in pure LF and ternary complex solutions before and after heat treatment were measured using CD spectroscopy. The CD spectra of LF and redispersed complex were measured using an AVIV-202-01 spectropolarimeter (Lakewood, NJ, USA) in the far-UV region (190-260 nm) at 25° C. To reduce the gain, samples were diluted to 0.02% (w/w) LF before measurement. Samples were analyzed in a quartz cell with a 1-mm path length. The obtained data were converted to molar ellipticity, [θ] (deg cm² dmol⁻¹), using the DichroWeb online processing platform. Pure gelatin showed no secondary structures according to its limited CD spectra signal; therefore, the CD spectra of the ternary complex was mainly referred to as the secondary structures of LF.

### 2.3.11 Intrinsic Fluorescence Analysis

The intrinsic fluorescence of LF and ternary complex samples before and after heat treatment was measured using a Shimadzu RF-6000 spectrophotometer (Shimadzu, Japan). Samples were measured in a 1-cm quartz cuvette at an excitation wavelength of 280 nm and the emission was monitored over the range of 310-400 nm; excitation/emission slit widths were both set at 10 nm.

### 2.3.12 Antimicrobial Activity Analysis

A strain of Staphylococcus aureus and a strain of Escherichia coli (E. coli) were used in this study as the target gram-positive bacteria and gram-negative bacteria, respectively. The S. aureus strain was isolated by the Animal Health Diagnostic Center of Cornell University (AHDC) from bovine feces. The E. coli (k12 Mg1655) strain was obtained from American Type Culture Collection (ACTT) (Freddolino et al., 2012). Frozen bacteria were first activated in Luria broth (LB) agar medium. Then a loop of a pure colony was transferred and incubated into fresh LB medium for 24 h at 37° C. for further antibacterial activity measurement.

The antibacterial activity measurement was performed on a 96-well microtiter plate using the UV absorbance method. First, the bacteria, S. aureus or E. coli, were diluted in LB broth 1000 times to make sure the absorbance of 100 μl bacteria broth was less than 0.04 at 625 nm. An increase in absorbance at 625 nm (OD 625 nm) was used to indicate the growth of bacteria. For a MIC (minimal inhibitory concentration) study, LF at different diluted concentrations (0.1-1% w/v) was used. A volume of 100 μL diluted bacterial broth and 100 μL LF solution was added to each well. Then, 100 μL bacteria broth with 100 μL PBS buffer was applied as the control. The minimal concentration of LF to inhibit 50% of bacterial growth (i.e., 50% of OD value in control) was used as the concentration for further antibacterial study of LF ternary complex study. Similarly, 100 μL of diluted bacterial broth and 100 μL of unheated and heated LF complex solution at the selected concentration (0.2%, w/v), based on the MIC study, were added to each well. The microtiter plate was incubated at 37° C. and the OD625 nm was measured to monitor the growth of bacteria at 0, 24, and 48 hours of incubation, with shaking 10 seconds before reading.

### 2.4 Data Analysis

The obtained data were presented as means and standard deviations of duplicates or triplicates and analyzed using Analysis of Variance (ANOVA). The difference between mean values was evaluated using the Tukey HSD comparison test (P<0.05). All the statistical analyses were performed using JMP Pro15 (SAS Institute, USA) and plotted by GraphPad Prism9 (GraphPad Software Inc., USA). The ternary plots were drawn by MATLAB (R2022a, MathWorks, Natick, MA, USA).

### 3. Results and Discussion

### 3.1 Effect of Biopolymer Ratios, PS, and Concentration on the Rormation of the Ternary Complex

FIG. 2 shows the effect of different polysaccharides and biopolymer ratios on the formation of ternary complex with LF and gelatin at pH 4. Charge density, structural characteristics, and approximate molecular weight of five polysaccharides were tabulated for reference (Table 1). In general, soy soluble polysaccharides (SSP) and gum Arabic (GA) are branched polysaccharides with a charge density lower than 0.3, while high methoxyl pectin (HMP), kappa carrageenan (Kappa), and iota carrageenan (Iota) are linear polysaccharides, with a charge density of 0.3˜0.6, 0.5, and 1, respectively. The total biopolymer concentration for each ternary system was the same (0.2 w/v %).

A ternary plot was used to design the mixing ratio of the three biopolymers (FIG. 2); the scale for the three axes indicates the mass ratio of the biopolymers being added to the ternary mixture. A darker grey color of the ternary mixtures at certain biopolymer ratios indicates higher turbidity (3-7) of mixtures, which further indicates a stronger interaction and complexation formation.

TABLE 1
Charge density, molecular weight, and supplier of five polysaccharides used
in current study. (Molecular weight information was provided by suppliers).

| | Charge | | Molecular | |
|---|---|---|---|---|
| Polysaccharides | density* | Structure description | weight | Supplier |
| Gum Arabic | <0.3 | Branch structure | 250 KDa | Colony |
| (GA) | | Main backbone: (1,3)-linked D- | | gums |
| | | galactopyranose units | | (USA) |
| | | Side-chain: D-galactopyranose, D- | | |
| | | arabofuranose, D- | | |
| | | galactopyranose, L- | | |
| | | rhamnopyranose | | |
| Soy soluble | <0.3 | Consist of long | 200-300 | Fuji Oil |
| polysaccharides | | rhamnogalacturonan (RG) and | KDa | (Japan) |
| (SSP) | | short homogalacturonan (GN) | | |
| | | Branched by β-1,4-galactan | | |
| | | α-1,3 or 1,5-arabinan chains | | |
| High methoxyl | 0.3~0.6 | α-(1,4)-linked galacturonic acid | 200 KDa | Tic gum |
| pectin | | residues | | (USA) |
| (HMP) | | Esterification degree: 69-77% | | |
| Kappa | 0.5 | Linear chain of alternating β-(1, | 150 KDa | Tic gum |
| carrageenan | | 3)-linked galactopyranos-4-sulfate | | (USA) |
| (Kappa) | | and α-(1,4)-linked 3,6- | | |
| | | anhydrogalactotose units | | |
| Iota carrageenan | 1 | Linear chain of alternating β-(1,3)- | 250 KDa | Tic gum |
| (Iota) | | linked galactopyranos-4-sulfate | | (USA) |
| | | and α-(1,4)-linked 3,6- | | |
| | | anhydrogalactotose-2-sulfate | | |
| | | units | | |

*Charge density in mol/mol monosaccharide

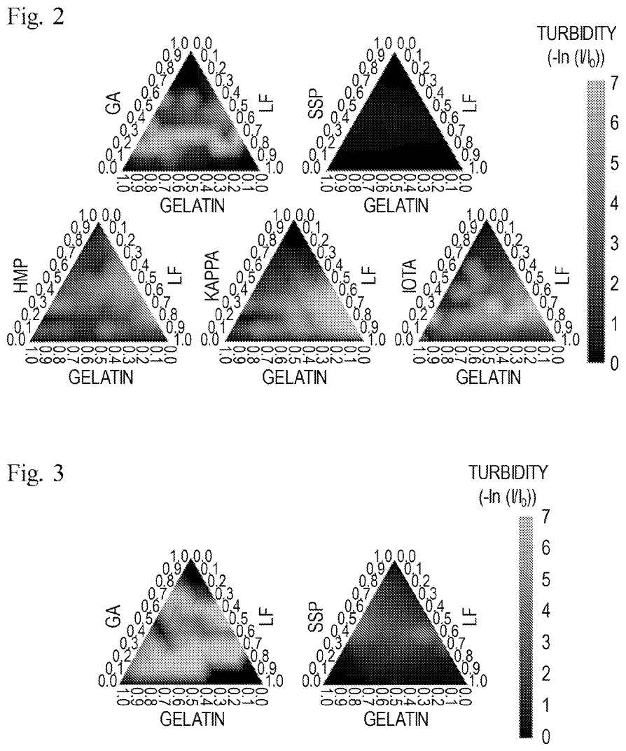

As shown in FIG. 2, different PS systems showed different maximum turbidity (Tₘₐₓ); complex formation was indicated when the turbidity was found to be greater than 2. The maximum turbidity represented the maximum extent of complexation and the strength of electrostatic interactions in the system, which is related to the relative amounts of oppositely charged groups in the individual solutions. Generally, PS (GA and SSP) with branch chains/structures showed a smaller Tₘₐₓ than PS (HMP, Kappa, and Iota carrageenan) with linear structures. The later systems also demonstrated a larger complexation formation area (when T>3), probably because of their larger negative charges and higher charge density. Considering the relatively low complexation formation in GA and SSP systems at 0.2%, an increased concentration (1%) was applied, as shown in FIG. 3.

For the GA-LF-G system, a complex was formed when the mixtures consisted of 60-80% LF and 20-40% GA (FIG. 2), while the concentration of gelatin seemed to have little effect. In general, SSP-LF-G mixtures showed a very limited complex formation in all studied conditions with relatively low turbidity, less than 3, across the range of mass ratios (FIG. 2). For the HMP-LF-G system, a complex was formed when the mixture consisted of 30-90% of LF, 30-90% of HMP, and 40-50% of gelatin (FIG. 2). For both Kappa-LF-G and Iota-LF-G mixtures, the complex formation range was shifted to 50-90% of LF, 50-90% of Kappa or Iota, and less than 50% of gelatin (FIG. 2). A higher gelatin concentration (>50%) seemed to disfavor the complex formation when linear polysaccharides were involved, showing a lower turbidity. While not wishing to be bound to any particular theory, the higher charge densities and greater number of negative charges carried by HMP, Kappa, and Iota may contribute to the increased ability of these polysaccharides to form complexes with LF at high mass ratios of LF in comparison to SSP and GA (Tables 1 and 2).

TABLE 2
Zeta potential of biopolymers at different
pH levels and concentrations (w/v %).

| Biopolymers | pH 4 (mv) | pH 7 (mv) |
|---|---|---|
| 0.2% LF | 21.03 ± 1.37 | 3.21 ± 0.58 |
| 0.2% Gelatin | 10.80 ± 0.70 | −0.97 ± 0.88 |
| 0.2% Gum Arabic | −13.27 ± 1.30 | −13.40 ± 2.59 |
| 0.2% SSP | −14.70 ± 0.30 | −19.10 ± 1.20 |
| 0.2% HMP | −28.6 ± 4.47 | −39.03 ± 3.19 |
| 0.2% Iota carrageenan | −40.3 ± 3.47 | −31.40 ± 1.04 |
| 0.2% Kappa carrageenan | −51.17 ± 1.58 | −53.63 ± 1.71 |
| 1% LF | 20.53 ± 0.45 | 1.98 ± 0.28 |
| 1% Gelatin | 7.96 ± 1.01 | −0.60 ± 0.19 |
| 1% Gum Arabic | −10.74 ± 0.51 | −20.73 ± 1.55 |
| 1% SSP | −8.06 ± 0.47 | −13.93 ± 0.95 |

Considering the relatively low turbidity in SSP and GA systems at the total concentration of 0.2 w/v %, an increased concentration of biopolymers (1 w/v %) in these two ternary systems was further studied (FIG. 3). Increased concentration enhanced the interactions as well as the Tₘₐₓ. especially in the GA ternary system. While it is expected that a higher concentration would promote a higher extent of complexation because of the increased density of opposite charges and the decreased molecular distances in solution, the extent of complexation was much more significant than expected in the GA-LF-G system and less apparent than expected in the SSP-LF-G system. The zeta-potential, representing the charge density, of GA and SSP was very similar in 0.2 w/v % or 1 w/v % concentrations (Table 2), therefore, without wishing to be bound to any particular theory, the differences in chain flexibility, between GA and SSP could be responsible for their observed differences in complex formation with LF and gelatin.

The ternary complex formed at pH 7 was also studied (data not shown). However, samples at all the biopolymers' ratios showed very low turbidity (<2), indicating limited complexation formation. Because LF and gelatin had limited positive charges at pH 7, the electrostatic interactions in all the biopolymers ratios were low. In total, the final selected concentration was 1% in GA and SSP ternary system and was 0.2% in HMP, Kappa, and Iota ternary system. The pH condition was fixed at pH 4 when all the biopolymers obtained comparatively high charges. According to these triangle turbidity plots, the biopolymers ratios with top high turbidity were selected as the conditions to form complex, as they indicated a higher level of complex formation in each system. Specifically, the selected biopolymers ratios were GA-LF-G 2-6-2, SSP-LF-G 4-5-1, HMP-LF-G 4-5-1, Kappa-LF-G 3-6-1, Iota-LF-G 2-6-2, respectively.

### 3.2 Zeta Potential and Particle Size of Ternary Complex

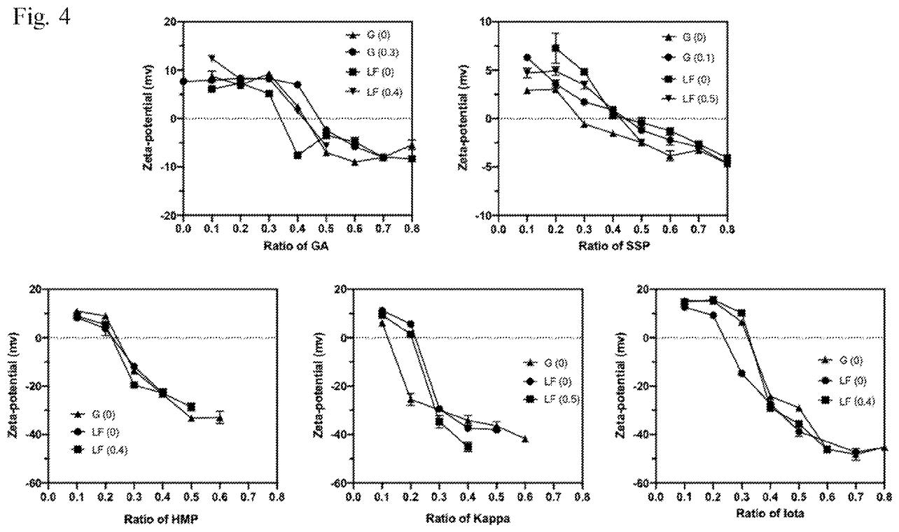

The zeta potential of ternary mixtures formed at pH 4 was selectively evaluated as a proof of concept. Typically, the ternary mixtures with fixed gelatin and LF ratio were chosen to study the effect of the addition of PS or LF on the zeta potentials of the ternary mixtures. As shown in FIG. 4, with the addition of negatively charged PS, the zeta potential of ternary mixtures gradually decreased from positive charges to zero and then further decreased into negative charges. Most importantly, the ratios when the overall zeta potentials close to zero were also the conditions that showed high turbidity as shown in FIGS. 2 and 3. This is because when the overall zeta potential reached zero, it means all the biopolymers had similar opposite charges on the surface. They tended to form complex due to electrostatic interactions and the repulsive interactions among biopolymers decreased as the system had the least charges, further facilizing the formation of a larger complex. The particle size of GA and Iota ternary mixtures at different ratios and pH conditions were also selectively measured and compared. As shown in Table 3, mixture samples of GA-LF-G 2-6-2 showed a large mean particle as the complex formed, while at pH 7, the same ratio showed a small mean size indicating limited complexation formation. Similarly, the samples of Iota-LF-G 2-6-2 showed an even larger size than the one obtained in GA-LF-G systems, possibly due to the higher charge density of Iota, although the total concentration of the Iota system was lower than the one in the GA system.

TABLE 3
Mean particle size of PS (GA/Iota)-LF-G mixtures
at pH 4 at various biopolymer ratios.

| Complex | Mean size (nm) | Complex | Mean size (nm) |
|---|---|---|---|
| GA-LF-G 7-0-3 pH 4 | 1676.33 ± 87.07 | Iota-LF-G 3-7-0 pH 4 | 4746.00 ± 999.85 |
| GA-LF-G 1-7-2 pH 4 | 129.03 ± 5.46 | Iota-LF-G 2-6-2 pH 4 | 2732.33 ± 720.15 |
| GA-LF-G 2-6-2 pH 4 | 1212.00 ± 187.97 | Iota-LF-G 1-8-1 pH 4 | 874.03 ± 453.96 |
| GA-LF-G 3-5-2 pH 4 | 312.77 ± 18.83 | Iota-LF-G 3-7-0 pH 7 | 203.07 ± 14.71 |
| GA-LF-G 2-6-2 pH 7 | 66.57 ± 2.28 | Iota-LF-G 2-6-2 pH 7 | 158.60 ± 13.98 |

### 3.3 Effect of Salt on the Complex Formation

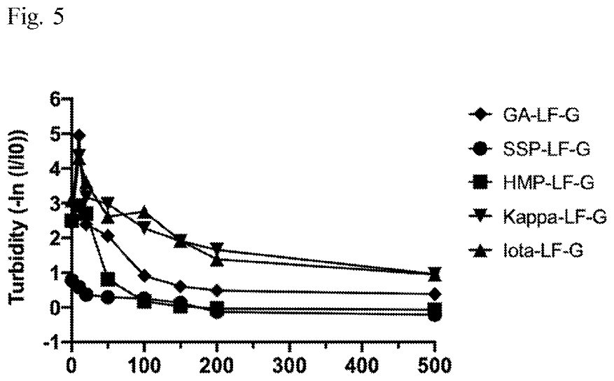

FIG. 5 shows the turbidity of ternary complex formed at different salt concentrations (up to 500 mM), at pH 4 and the ratios obtained from the above study. Salt generally showed a negative effect on ternary complex formation especially at a high concentration (>100 mM) since salt could weaken or screen out the electrostatic interactions between these polymers. An interesting finding is that the turbidity of the GA-LF-G ternary complex was increased at a low salt concentration (0-50 mM), and then gradually decreased with the increase of salt concentration. Generally, the ternary complex formed by SSP and HMP are more sensitive to salt, the turbidity being close to zero when the salt concentration is higher than 100 mM (which is also called critical salt concentration). The complex formed by GA was less sensitive to salt which had a critical salt concentration of around 200 mM. The complex formed by Kappa and Iota carrageenan showed the least sensitivity to salt, as they can retain their turbidity greater than 1 even at a salt concentration of 500 mM. Overall, most of the ternary complex can be retained at a salt concentration lower than 50 mM, preferably lower than 30 mM.

### 3.4 Complex Yield, Complexation Efficiency, and Loading Ratio of LF in the Ternary Complex

After obtaining the desired conditions for forming a ternary complex, the ternary mixture solutions were centrifuged and the supernatant, with un-complexed biopolymers, was removed. The resulting pellet, with complexed biopolymers, was then freeze-dried. The average yield, complexation efficiency, and final mass loading ratio of LF in the freeze-dried complex were measured (Table 4). The ternary mixtures formed with the linear, highly charged PS Kappa and Iota carrageenan showed the highest yield of complex (70-80%) with the highest complexation efficiency (96%). Conversely, the mixtures formed with the linear, less charged PS HMP provided a moderate yield of complex (˜40%) and a moderate complexation efficiency (52%). The ternary mixtures formed with the branched, even less charged PS, GA and SSP, showed a lower yield (40% and 17%) and a moderate to low complexation efficiency (50% and 20%). A high complexation efficiency (96%) in Kappa-LF-G and Iota-LF-G mixtures indicated that almost all LF present in the mixtures was incorporated into the complex, while GA/HMP-LF-G mixtures only incorporated ˜50% of the available LF. Generally, the results showed that the complex formed with the linear and more highly charged PS, Kappa, and Iota, showed a higher yield and complexation efficiency than those formed with the more neutral PS, HMP and GA. This result is consistent with the turbidity results that highly charged polysaccharides, Kappa, and Iota carrageenan, demonstrated a higher turbidity and complex formation with LF and gelatin, indicating stronger interactions in these ternary complexes.

TABLE 4
Complexation efficiency (%), yield (%), and LF loading ratio
(%) of the ternary complexes at desired mixing ratios.

| | Complexation | | Loading |
|---|---|---|---|
| Ternary complex | Efficiency (%) | Yield (%) | ratio (%) |
| GA-LF-G (2:6:2) | 46.4 ± 5.03% | 39.30 ± 13.64% | 52.04 ± 2.79% |
| SSP-LF-G (4:5:1) | 17.04 ± 11.26% | 16.53 ± 12.06% | 40.85 ± 0.79% |
| HMP-LF-G (4:5:1) | 52.50 ± 10.61% | 40.63 ± 3.36% | 42.05 ± 2.87% |
| Kappa-LF-G (2:6:2) | 96.50 ± 4.94% | 71.38 ± 1.95% | 54.28 ± 3.37% |
| Iota-LF-G (2:6:2) | 96.50 ± 4.94% | 81.10 ± 1.28% | 67.55 ± 5.72% |

The final mass ratio of LF in the freeze-dried complex was quantified using three different methods: Bradford assay, Bicinchoninic acid (BCA) assay, and HPLC. Based on these three methods, the mass ratio of LF in a complex including GA, SSP, or HMP was in the range of 40-56% (w/w). Due to the interactions of the Bradford agent with the sulfate groups of carrenganan, the mass ratio of LF in Kappa/Iota carrageenan ternary complex was underestimated by the Bradford assay. HPLC analysis was also restricted to obtaining the LF peak from Kappa/Iota carrageenan ternary complex because of the strong negative charges from the sulfate groups of Kappa/Iota carrageenan. Therefore, the mass ratio of LF in Kappa/Iota carrageenan ternary complex as measured by the BCA method was more reliable, which was around 68-75% (w/w). The LF loading ratio in the GA complex was ˜52%, the loading in HMP complex was ˜40-42%, while in the Kappa and Iota carrageenan complex was around 54-68% (Table 4-). The relatively high mass ratio of LF in a carrageenan ternary complex can be expected as those two polysaccharides carry much higher negative charges to form a complex with LF through electrostatic interactions.

### 3.5 Microscopy of Formed Complex

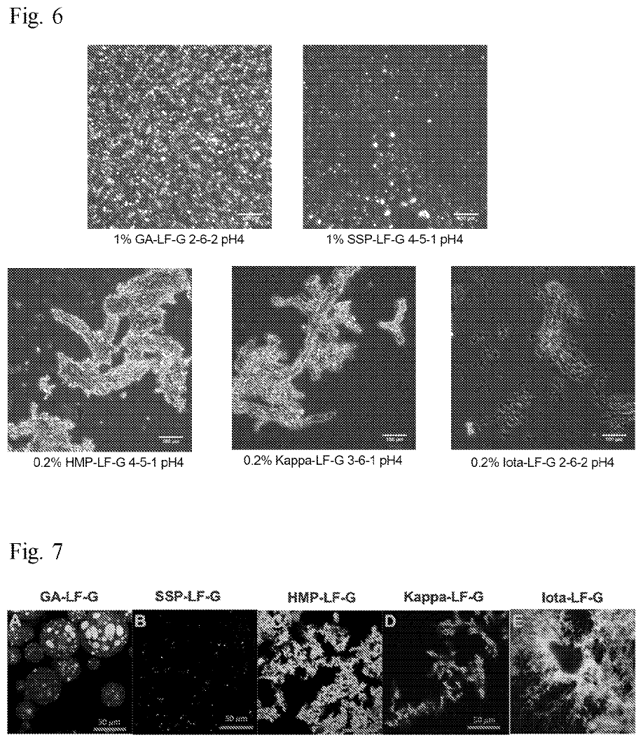

The optical microscopy of the ternary complex at selected conditions (pH, concentration and ratios) was investigated and shown in FIG. 6. Interestingly, different complex structures were formed in those ternary systems. GA and SSP systems showed spherical structures to some extent, while HMP, Kappa, and Iota systems showed irregular polymeric network structures. The former complex indicated the formation of coacervate complex while the latter showed an interpolymeric complex. Generally speaking, coacervate and complex development follows the same initial path. Initially soluble intrapolymeric complex formed at specific pH, ratios, and ionic strength, then as the biopolymer ratios continue to approach the conditions when the overall charges reach zero, soluble complex began to interact with each other to form interpolymeric complex followed by bulk phase separation. Generally, coacervates are formed when PS or protein poses a low charge density or very flexible backbone, such as gelatin, acacia gum, and gum arabic. Whereas interpolymeric complex, also called coprecipitates forms when the PS or the protein in the system is highly charged and/or has a very stiff linear structure, such as carrageenan, gellan gum, or xanthan gum. The formation of the intrapolymeric complex may be related to a larger binding affinity between biopolymers which results in strong interaction.

The confocal microscopy of SSP, GA, HMP, Kappa, and Iota ternary systems with FITC labeled LF (in light gray) is shown in FIG. 7 and SEM images are shown in Panels A1-E1 and A2-E2 of FIG. 17. Interestingly, the GA-LF-G ternary complex displayed as multiphase coacervate droplets, which means multiple coacervate droplets were inside a larger coacervate droplet. Furthermore, LF (FTIC labeled in green) seemed to be located in the inner phase coacervates. The SEM of the GA-LF-G complexes after overnight vacuum drying (Panels A1 and A2 of FIG. 17) showed the GA-LF-G complex as a compressed flat particle due to the water evaporation, indicating the liquid-like properties of coacervates. In confocal images, a scattering of SSP-LF-G nano-sized coacervate particles can be seen, indicative of the low yield (8%) of SSP-LF-G complexes formed, while the HMP-LF-G, Kappa-LF-G, and Iota-LF-G complex all showed the characteristic morphology associated with dense interpolymeric network structures (FIG. 7). The SEM images confirmed the interpolymeric complex structure of the ternary complex using these three linear polysaccharides, HMP, Kappa and Iota (Panels C1, C2; D1, D2; and E1, E2 of FIG. 17).

Although multiphase coacervate droplets have been recently reported in biological systems, to the best of our knowledge, it is the first time that this kind of phenomenon was observed in food-grade biopolymers, specifically protein-polysaccharides systems. While not wishing to be bound by any particular theory, it believed that the major driving forces to form a coacervate complex in multiphase droplets are the different critical salt concentrations and densities. The SSP-LF-G ternary complex showed as the conventional single phase coacervate droplet. Regarding the HMP and Iota ternary systems, the confocal microscopy images were similar to the optical microscopy images, which indicated the interpolymeric complex structure of ternary complexes including linear polysaccharides.

### 3.6 Thermal Stability of LF in Ternary Complex

### 3.6.1 Optical Images, Turbidity and Particle Size of LF Ternary Complex After Thermal Treatment

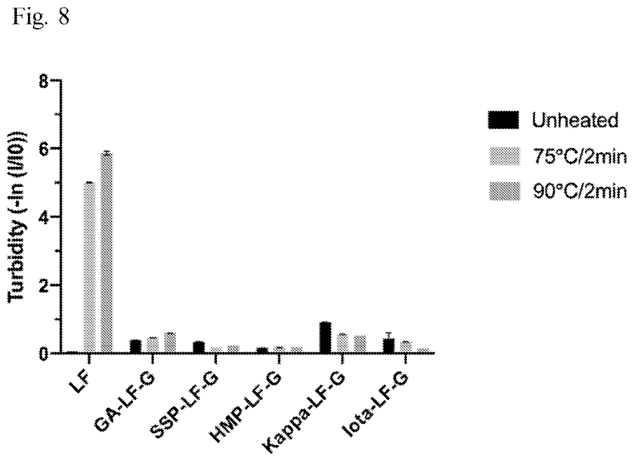

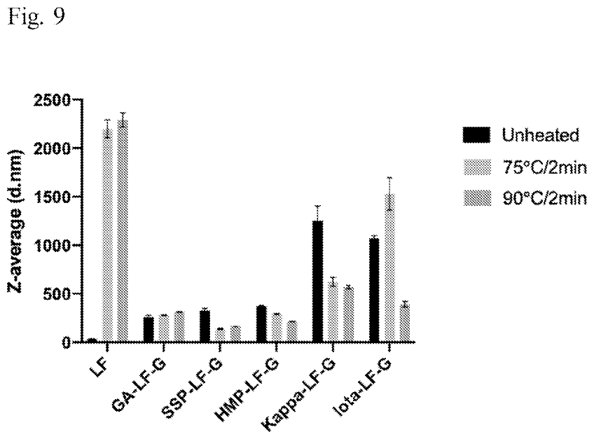

LF is known to be easily denatured under thermal processing conditions at neutral pH. Thus, to exam the thermal stability of LF in complexed samples, the freeze-dried complex samples were re-dispersed at 0.2% (m/v) in 10 mM PBS buffer at pH 7 and then thermally treated at pasteurization conditions (75° C./2 min and 90° C./2 min). Pure LF was easily denatured and aggregated in PBS buffer at pH 7. Furthermore, pure LF tended to aggregate to a higher extent at 90° C./2 min than 75° C./2 min with the former condition showing a more turbid solution. Compared to pure LF, no matter in which type of complex solutions, LF complex demonstrated much clearer solutions after heating treatment. This can be more directly observed in the turbidity chart in FIG. 8 that pure LF showed a large increase of turbidity from <0.5 to >4 in PBS buffer after being heated, while the turbidity increase of LF in complex samples was very limited (<1). FIG. 9 shows the particle size changes for LF and redispersed complex solutions after heating treatment. The aggregation of LF proteins caused a significant increase in particle size from <100 nm to >2000 nm, while complex solutions showed only a slight change in particle size. The Kappa and Iota complex even showed a decrease in particle size probably because heating promotes the solubilization of the formed complex by breaking large complexes into smaller soluble complexes. As pointed out previously, the complex formed by kappa and iota carrageenan showed strong interactions between LF and G, which enabled them to form larger particles and thus showed a lower solubility in solutions. While not wishing to be bound to any particular theory, it is postulated that the heating process could induce the breakdown of the large ternary complexes into smaller complex particles thus improving their solubility and reducing their overall particle size. They were still considered complexes, however, as their particle sizes were still larger than the particle size of native LF, which was measured to be ˜60 nm.

### 3.6.2 SDS-PAGE of LF and Ternary Complex After Thermal Treatment

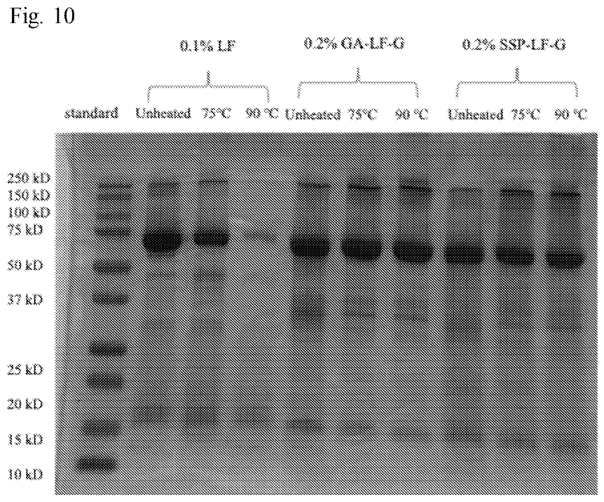

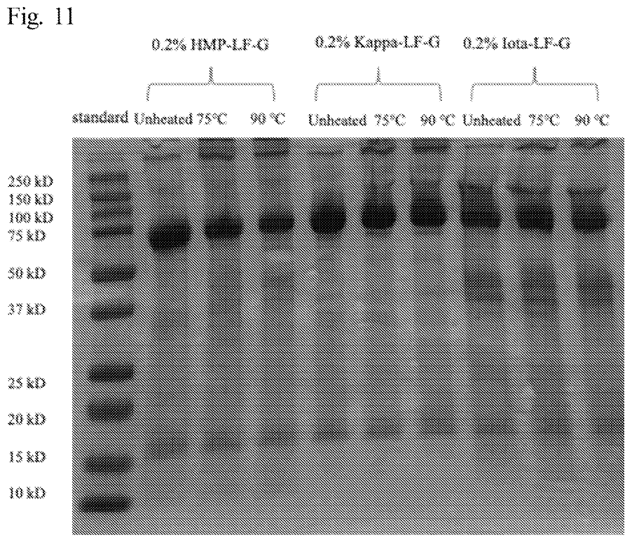

The SDS-PAGE of LF in pure LF and ternary complex after thermal treatment in PBS buffer solutions was analyzed and shown in FIGS. 10-11. In pure native LF, a clear band was shown around 75 kDa, which was the band where LF protein was presented. After being heated at 75° C./2 min and 90° C./2 min, the density of the LF band become significantly lighter compared to unheated LF, because of thermal degradation. This result is consistent with the observed change in the CD signal and the intrinsic fluorescence peak intensity of pure LF, described below. In complex samples, the density of LF band was also decreased, but to a much lesser extent compared to pure LF. The complex of GA-LF-G showed neglectable changes in the LF band after heating treatment. Other PS ternary complexes showed slightly more degradation of LF compared to the GA-LF-G complex. For the Kappa and Iota complexes, there was a remarkable tailing of the band in the SDA-PAGE; while not wishing to be bound to any particular theory, this was probably due to the stronger binding interactions between LF and the negative sulfate groups of carrageenan and more stiff structure of the complex, which affects the separation of LF molecules in the SDS-PAGE.

### 3.6.3 LF Retention Percentage in Ternary Complexes After Thermal Treatment by HPLC Analysis

To mathematically quantify the amount or the ratio of LF degraded or retained in the complex after thermal treatment, an HPLC method for LF analysis was developed and applied. The ternary complex of GA-LF-G, SSP-LF-G, and HMP-LF-G were focused on before and after thermal treatment because the strong binding interactions between LF and the Kappa and Iota carrageenans interfered with the method. The LF retention percentage in pure LF at different concentrations was analyzed first. LF retention percentage in the ternary complex was measured and compared with the corresponding binary complexes. As shown in Table 5, LF in the concentrations of 0.05-0.2% (w/v) (i.e., 0.5-2 mg/mL) in PBS buffer (pH 7) had only half of LF retained after being heated at 75° C./2 min. After being heated at 90° C./2 min, almost all LF was denatured, with only less than 10% of LF retained in all studied concentrations.

A comparison between the LF ternary complexes and the common binary complex (LF with GA, SSP and HMP) was also conducted. Binary complex showed an increased LF retention percentage which was 70-80% and 50-60% after being heated at 75° C./2 min and 90° C./2 min, respectively. Compared to the binary complex, ternary complexes demonstrated enhanced LF stability during thermal treatment. The LF in ternary complexes was only slightly degraded with 80-100% retention after being heated at 75° C./2 min. Even being heated at 90° C./2 min, there was 70-99% retention of LF, which is seven to nine times higher retention compared to pure LF samples and almost one time more retention compared to binary complexes. In the GA-LF-G ternary complex, LF was almost not degraded at all in both heating conditions, showing the highest LF thermal stability among all ternary complex samples. While not wishing to be bound to any particular theory, the enhanced LF thermal stability in ternary complexes described herein may be attributed to the additional complexation interaction from the additional biopolymers. In addition to the interactions between LF and the PS, without being bound to any particular theory, the interaction between gelatin and the PS may enhance the interpolymeric interactions within the complex, strengthening the protection around LF and restricting protein structural changes during thermal treatment.

TABLE 5
LF retention percentage of thermal-treated LF and binary/ternary
complex in PBS buffer (10 mM, pH 7) quantified by HPLC analysis.

| LF retention % after | | LF retention % after | |
|---|---|---|---|
| heating at 75° C./2 min | | heating at 90° C./2 min | |
| 0.2% LF 75 | 53.71 ± 1.80% | 0.2% LF 90 | 4.74 ± 0.64% |
| 0.1% LF 75 | 52.83 ± 5.54% | 0.1% LF 90 | 6.76 ± 0.96% |
| 0.05% LF 75 | 58.56 ± 2.20% | 0.05% LF 90 | 0.78 ± 0.68% |
| GA-LF 75 | 82.17 ± 4.58% | GA-LF 90 | 56.70 ± 5.20% |
| SSP-LF 75 | 74.47 ± 2.19% | SSP-LF 90 | 52.90 ± 0.44% |
| HMP-LF 75 | 74.19 ± 2.52% | HMP-LF 90 | 57.17 ± 2.25% |
| GA-LF-G 75 | 100.24 ± 0.30% | GA-LF-G 90 | 99.07 ± 1.15% |
| SSP-LF-G 75 | 91.47 ± 0.82% | SSP-LF-G 90 | 71.20 ± 4.79% |
| HMP-LF-G 75 | 80.91 ± 0.46% | HMP-LF-G 90 | 73.17 ± 1.47% |

### 3.6.4 Structure Changes of LF in Ternary Complex After Thermal Treatment

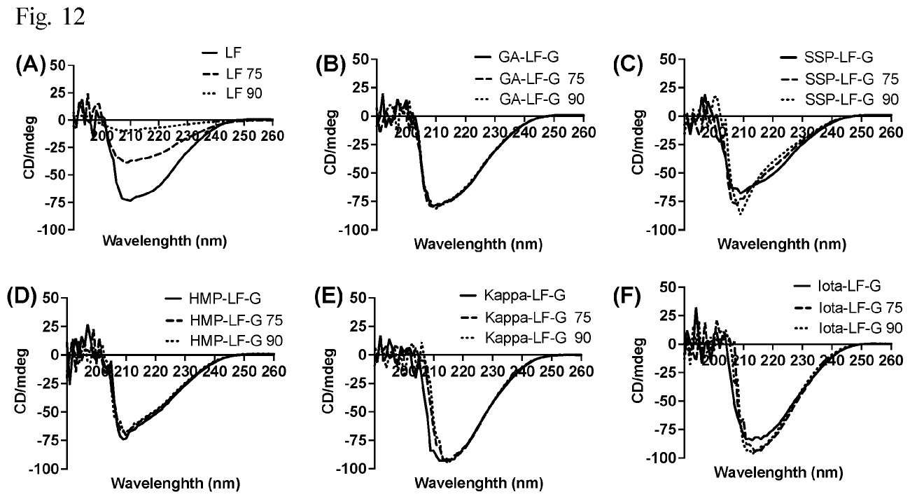

The CD spectroscopy of LF ternary before and after thermal treatment was also measured to look into secondary structure changes of LF during thermal treatment. As shown in FIG. 12, pure native LF had a positive peak at 196 nm and a negative peak at 210 nm, indicating the beta and alpha structure of the LF protein. LF is reported to consist of 16-20% of α-helix, 33-42% of β-strands, 10-12% of β-turns, and 30-34% of unordered structures (Lin et al., 2022; Wang et al., 2017). After heating, pure LF showed a significant decrease in peak intensity at 210 nm, indicating the loss of alpha-helix structure. The higher the heating temperature, the larger the decrease of peak intensity. Particularly after heating at 90° C./2 min, the CD spectroscopy signal and the peak intensity of LF were very low indicating a high degree of degradation and loss of alpha-helix of LF. However, the CD spectroscopy for all the complex solutions was barely changed even after being heated up to 90° C./2 min, demonstrating that the LF secondary structures were well preserved in the complex solutions during heating process (Panels B-F of FIG. 12). Particularly, the CD spectrum of GA-LF-G complex solutions was barely changed after thermal treatment, showing a high degree of preservation of LF native structures (Panel B of FIG. 12). Our previous work also found improved thermal stability of LF secondary structures by complexation with negatively charged soluble soy polysaccharides (Lin et al., 2022); however, such limited change of secondary structures of LF demonstrated in current GA-LF-G samples has not been reported previously.

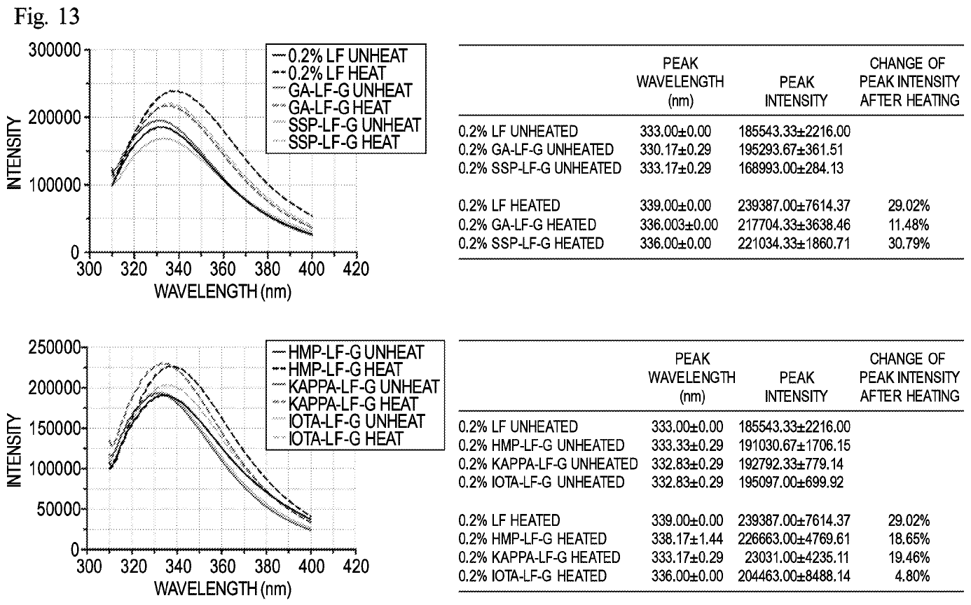

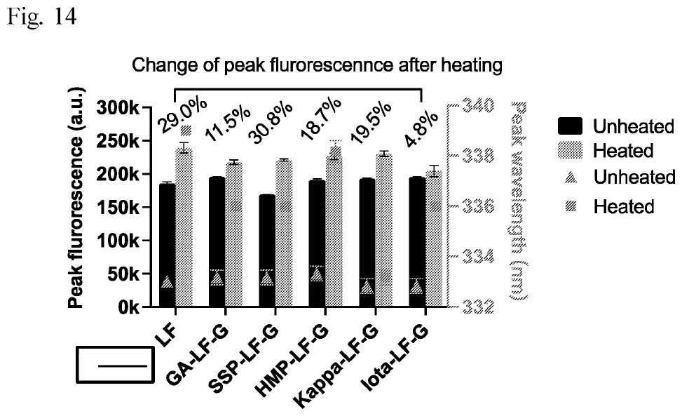

To further understand the structural changes of LF in the system after the heating process, the intrinsic fluorescence intensity of unheated and heated samples was investigated, as shown in FIGS. 13 and 14. The fluorescence emission maximum peak of pure LF showed a red shift from 333 to 339 nm in the fluorescence spectrum, which is typical for a tryptophan residue in the unfolded protein, thus indicating the unfolding of LF during the heating process. Furthermore, the peak intensity of LF was increased by 29% after being heated, indicating reduced quenching to tryptophan upon the unfolding of LF during the heating process (FIG. 14). After thermal treatment, the emission peak of complex samples showed a less red shift indicating less unfolding of the protein structure (FIG. 14). The increase of peak intensity in complex samples after heating was also smaller (4.8-18.7%) than that in pure LF, except for SSP (FIG. 14). Overall, the intrinsic fluorescence results showed that, compared to pure LF, LF in the ternary complex (especially GA, HMP, Kappa, or Iota system) retained more native protein structures with less unfolding after the heating process.

TABLE 6
LF retention ratio in LF and LF ternary complexes in PBS
buffer (10 mM, pH 7) after thermal treatment at 145°
C. for a specified amount of time as quantified by HPLC.

| Samples | Time (s) | LF retention % after heating |
|---|---|---|
| 0.1 w/v % LF | 2 | 100.49 ± 2.85% ^{ab} |
| 0.1 w/v % LF | 10 | 97.08 ± 2.39% ^{bcd} |
| 0.1 w/v % LF | 30 | 16.48 ± 0.28% ^{g} |
| 0.1 w/v % LF | 60 | 7.76 ± 0.11% ^{h} |
| 0.2 w/v % GA-LF-G | 2 | 100.90 ± 0.38%^{a} |
| 0.2 w/v % GA-LF-G | 10 | 99.94 ± 0.70% ^{ab} |
| 0.2 w/v % GA-LF-G | 30 | 98.34 ± 1.16% ^{abc} |
| 0.2 w/v % GA-LF-G | 60 | 84.06 ± 1.32% ^{e} |
| 0.2 w/v % HMP-LF-G | 2 | 100.72 ± 0.24% ^{ab} |
| 0.2 w/v % HMP-LF-G | 10 | 95.55 ± 0.44% ^{cd} |
| 0.2 w/v % HMP-LF-G | 30 | 93.83 ± 0.54% ^{d} |
| 0.2 w/v % HMP-LF-G | 60 | 80.05 ± 0.82% ^{f} |

Different letter superscripts indicate a statistically significant difference between the mean values (P < 0.05), compared using Tukey-Kramer HSD.

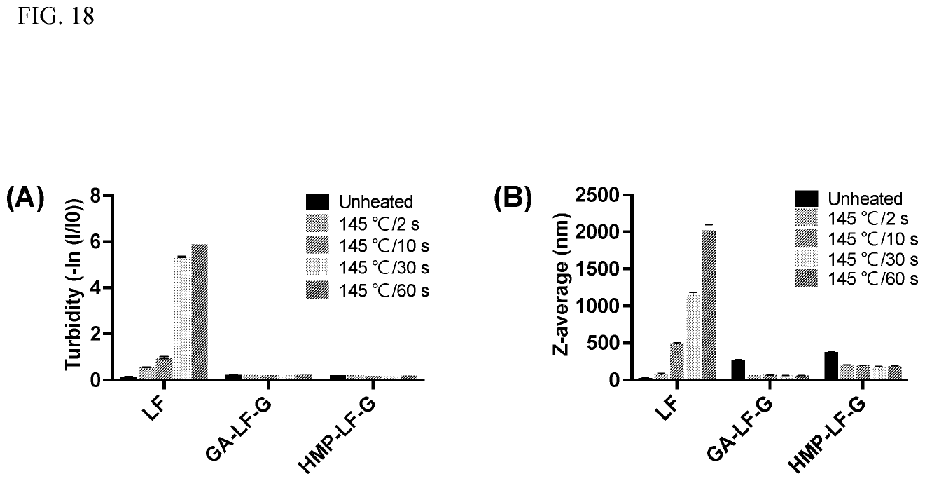

Considering the common usage of ultra-high temperature (UHT) treatment in industrial food processing, the thermal stability of LF under a higher heating temperature at ˜145° C. for 0-60 s was investigated (Table 6). LF solutions started to show observable denaturation or loss of native LF after being heated in oil bath for 30 s with 16.5% retention, and by 60 s with only 7.8% retention as found by HPLC analysis. The GA-LF-G ternary complex showed little change after heating for 30 s at 145° C., while the HMP-LF-G ternary complexes were less resilient only remaining intact for 10 s. Both complexes showed a significant decrease of LF retention (84.1% for GA-LF-G and 80.0% for HMP-LF-G) after 60 s of UHT treatment. The turbidity and particle size changes of the LF and ternary complex solutions were also analyzed, showing consistent results with the LF retention analysis (FIG. 18). Specifically, LF solution showed a significant increase of turbidity (Panel A of FIG. 18) and mean particle size (Panel B of FIG. 18) after 30 s treatment, while ternary complexes showed neglectable changes of turbidity and particle size after 60 s at 145° C. The ternary complexes maintained the stability of LF even under high temperature conditions.

### 3.7 Antimicrobial Properties of LF in Ternary Complex Before and After Thermal Treatment

LF is known to exhibit anti-bacterial capacity against both gram-positive and gram-negative bacteria, retaining these properties while increasing heat stability is desired. The mechanism of the antibacterial activity of LF is currently not well understood. The antimicrobial properties of LF were evaluated in pure and complex LF samples before and after heating treatment. This part of the study aims to examine whether complexation will influence the functionality of LF and whether it can preserve this capacity after heating treatment. Antimicrobial capacity was chosen as a representative biological functionality of LF, as it can be performed easily and relatively safe in most biological labs.

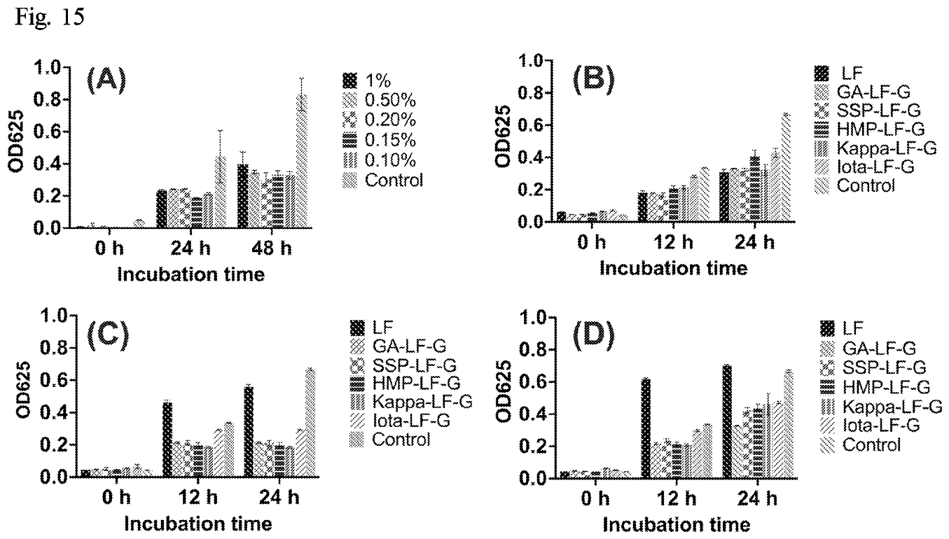

S. aureus was chosen as the target Gram-positive bacteria considering it is one of the most common pathogens in dairy foods. A MIC (minimal inhibitory concentration) study of LF on S. aureus was first conducted, which is defined as the minimal concentration of LF that was able to inhibit half of the bacterial growth compared to the control group (Matijašić et al., 2020). As shown in Panel A of FIG. 15, the MIC of LF on S. aureus is 0.1% since the OD625 nm value in LF of 0.1% was only half of the value in the control sample, which means 0.1% of LF was enough to inhibit 50% of bacteria growth. In redispersed ternary complex solutions (0.2% w/v), the concentration of LF was around 0.1-0.14% thus suitable for the antibacterial tests. Panels B-D of FIG. 15 show the OD625 nm value of unheated samples or the OD625 nm value of heated samples after 75° C./2 min or 90° C./2 min. Pure polysaccharides and gelatin did not exhibit any antibacterial effect and even promoted bacterial growth (data not shown) as they could be consumed by bacteria as energy sources. Our initial ternary complexes showed a similar OD625 as that of native LF, confirming that the LF functionality was well retained after LF formed ternary complexes before heat treatments.

For unheated samples (Panel B of FIG. 15), LF clearly demonstrated antibacterial activity after 24 h of incubation with half amount of OD625 nm value compared to the control. The ternary complex samples also showed an inhibitory effect; however, it was less effective compared to pure LF. While not wishing to be bound to any particular theory, this may be because LF was complexed/encapsulated in the ternary complex, which may affect the interaction between LF and bacteria and further influence the inhibitory effect to some extent.

After being thermally treated at 75° C./2 min (Panel C of FIG. 15), pure LF lost its inhibitory effect on bacteria and even promoted bacterial growth, probably since bacteria can utilize the degraded LF fragments as energy sources. However, the thermally treated ternary complex demonstrated a significant inhibitory effect, with about half of OD625 nm value compared to the control. This effect was sustained until 48h of incubation. As shown in FIG. 15, thermally treated ternary complexes produced demonstrated an increased antibacterial effect. While not wishing to be bound to any particular theory, the improved antibacterial effect after heat treatment at 75° C. may be attributed to the degradation of the ternary complex structures and the partial exposure or release of LF during the heating process, which promoted interactions between LF and the bacteria (Panel C of FIG. 15).

Nevertheless, at 90° C. the partial denaturation of LF caused a significant decrease in the antibacterial activity of the ternary complexes (Panel D of FIG. 15). It seems the formed ternary complex demonstrated a temperature-responsive release ability of LF. During thermal treatment, LF may be gradually released from the complex and still maintain its native structure and antibacterial capacity. In 90° C./2 min thermally treated samples (Panel D of FIG. 15), the loss of inhibitory effect of LF and presence of bacterial growth was even more significant as it showed an even higher OD625 nm value compared to75° C./2 min-treated counterparts. However, the ternary complex still showed about 50% inhibition on bacterial growth with half of the OD 625 nm value of control samples, especially in GA, SSP and HMP ternary complexes. The ternary complex formed by Kappa and Iota carrageenan showed a lower antibacterial capacity (i.e., a higher OD value) compared to other three PS, possibly due to their strong binding with LF thus restricting the release of LF to carry out antibacterial effect. Overall, the results demonstrated that pure LF had significantly reduced antibacterial capacity after heating, while antibacterial capacity was well preserved in the ternary complexes tested.

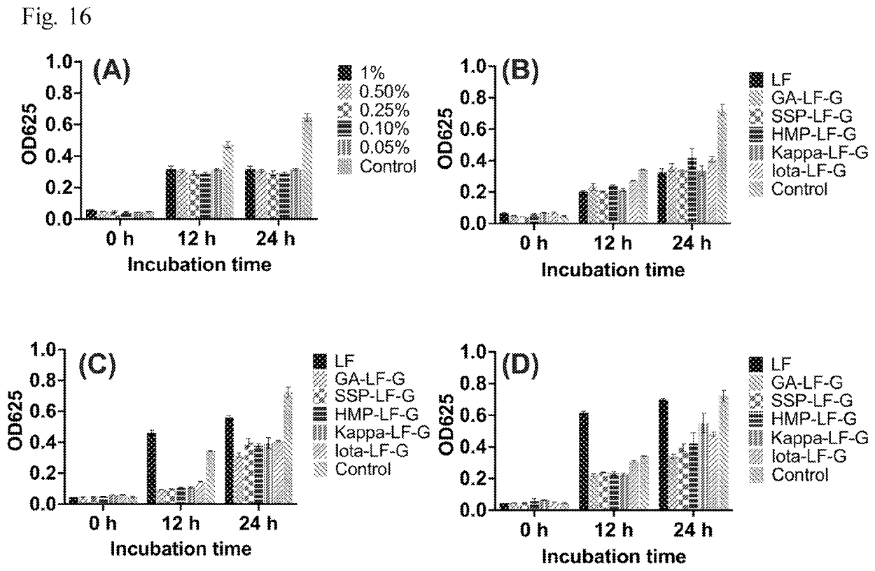

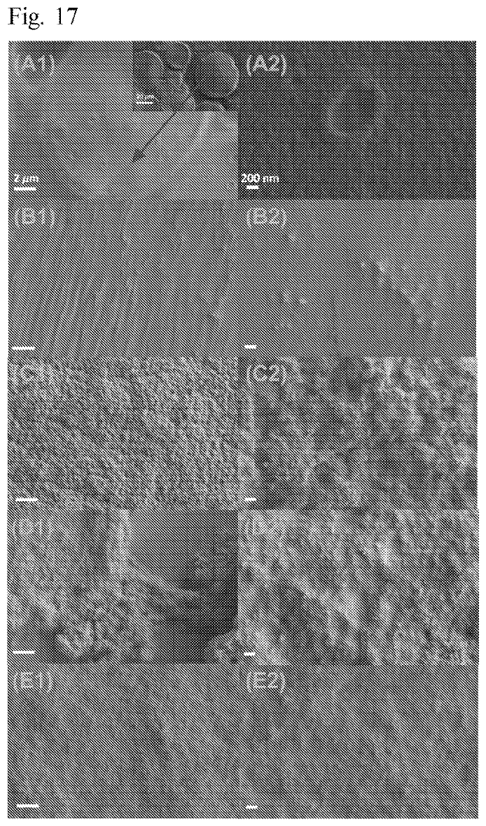

E. coli is a common spoilage Gram-negative bacterium in food products, therefore the antibacterial activity of LF and our ternary complexes against E. coli was also tested. Within 24 h incubation time, OD625 of E. coli in control samples was slightly higher than that of S. aureus, representing the generally faster bacterial growth rate of E. coli than S. aureus (Panel A of FIG. 15 and Panel A of FIG. 16). Although the MIC of LF on E. coli was similar to the MIC of LF on S. aureus, around 0.1 w/v %, the antibacterial effect of LF on E. coli was weaker than that on S. aureus since the E. coli samples all showed a higher OD625 value after a 24 h incubation. Higher environmental resistance of Gram-negative bacteria than Gram-positive bacteria is attributed to the protective, impenetrable cell wall of Gram-negative bacteria which is surrounded by an outer membrane that Gram-positive bacteria lack (Breijyeh et al., 2020). For unheated samples, both LF and LF ternary complex demonstrated antibacterial effect on E. coli within a 24 h incubation (Panel B of FIG. 16). Similar to the results on S. aureus, heated LF samples lose antibacterial effect on E. coli after heat treatment due to the heat denaturation of LF (Panels C and D of FIG. 16), while the ternary complexes retained antibacterial effect showing significantly lower OD625 values than the control.

Overall, the conditions (ratio, pH, and concentration) to form a ternary complex of LF, gelatin, and different polysaccharides were identified for each ternary system according to the turbidity plots. Branched, less charged Gum Arabic and SSP formed a coacervate complex with LF and gelatin at a total concentration of 1%. A further higher concentration may be also applicable in both systems considering the relatively high solubility and low viscosity of GA and SSP. Linear, highly charged HMP, Kappa and Iota formed an interpolymeric complex with LF and gelatin at a total concentration of 0.2%. An appropriate hardening and/or cross-linking procedure can enhance the yield of the formed complex. Further freeze-drying and/or spray drying process can produce coacervate complex into microcapsules. The formed complex samples were shown to retain most of the native LF structures even after being heated up to 90° C./2 min based on HPLC analysis and CD spectroscopy. Also, the ternary complexes including GA, SSP, or HMP were shown to maintain the antimicrobial capacity (e.g., were able to inhibit bacteria growth by about half of the amount, compared to control, as indicated by the OD 625 nm value) of LF after pasteurization conditions (75° C./2 min and 90° C./2 min) at a neutral pH condition.

### Example 2

In this study, we fabricated thermal-stable lactoferrin (LF) ternary complexes through electrostatic interactions under lab-scale and industrial-scale processes.

The formed LF complexes showed improved thermal stability compared to individual LF under High-Temperature Short Time (HTST)/Ultra-High Temperature (UHT) processing in different matrices including water, skim milk, and acid whey beverages. The retention rate of LF after thermal treatment was quantified by HPLC and ELISA analysis and the protein secondary structure was further confirmed through circular dichroism spectroscopy. The bioactivity of LF including antibacterial (E. coli and S. aureus) and antiviral (Coronavirus) activity were well retained in the complexes after thermal treatment.

During storage, LF complex ingredients demonstrated good solubility, LF retention rate, and bioactivity at 25° C. for 12 months. The formed LF complexes in different food models (water, skim milk, and acid whey beverage) displayed stable physiochemical properties (e.g., turbidity and particle size) at the storage conditions of 4° C. for 12 months and 25° C. for 6 months and delivered a minimal undesirable sensory property.

The foregoing is illustrative of the present invention and is not to be construed as limiting thereof. The invention is defined by the following claims, with equivalents of the claims to be included therein.

That which is claimed is:

1. A complex comprising:
a protein;
an anionic biopolymer; and
a cationic biopolymer;
wherein the protein, anionic biopolymer, and cationic biopolymer are associated via electrostatic interactions.

2. The complex of claim 1, wherein the complex has a zeta potential of about −5 mV to about +5 mV, optionally wherein the complex has a net negative charge at a pH of about 6.5 to about 7.5.

3. The complex of claim 1 or 2, wherein the complex comprises the protein in an amount of about 30% to about 85% w/w, the cationic biopolymer in an amount of about 5% to about 40% by w/w, and the anionic biopolymer in an amount of about 5% to about 40% w/w, optionally wherein the complex comprises the protein in an amount of about 40% to about 75% w/w, the cationic biopolymer in an amount of about 10% to about 30% by w/w, and the anionic biopolymer in an amount of about 10% to about 30% w/w.

4. The complex of any preceding claim, wherein the protein has a net positive charge at a pH of about 3, 3.5, or 4 to about 4.5, 5, 6, 7, or 8, optionally wherein the protein at a pH of about 3, 3.5, or 4 to about 4.5 or 5 has a zeta potential of greater than about +10 mV to about +30 mV.

5. The complex of any preceding claim, wherein the protein has a globular structure and/or is a dairy protein.

6. The complex of any preceding claim, wherein the protein is soluble in water at a pH of less than about 8.

7. The complex of any preceding claim, wherein the protein is lactoferrin, alpha lactalbumin, lysozyme, and/or osteopontin, optionally wherein the protein has an amino acid sequence having at least 70%, 75%, 80%, 85%, 90%, 95%, 96%, 97%, 98%, 99%, or more sequence identity to one or more of SEQ ID NOs: 1-5.

8. The complex of any proceeding claim, wherein the cationic biopolymer has a net positive charge at a pH of about 3, 3.5, or 4 to about 4.5 or 5, optionally wherein the cationic biopolymer at a pH of about 3, 3.5, or 4 to about 4.5 or 5 has a zeta potential of greater than +10 mV to about +30 mV.

9. The complex of any preceding claim, wherein the cationic biopolymer has a pI and/or a pKa of about 7 or more.

10. The complex of any preceding claim, wherein the cationic biopolymer is selected from a gelatin, chitosan, lysozyme, and/or a polyamino acid, optionally wherein the cationic biopolymer is a gelatin.

11. The complex of any proceeding claim, wherein the anionic biopolymer has a net negative charge at a pH of about 3, 3.5, or 4 to about 4.5 or 5, optionally wherein the anionic biopolymer at a pH of about 3, 3.5, or 4 to about 4.5 or 5 has a zeta potential of less than −10 mV to about −50 mV.

12. The complex of any proceeding claim, wherein the anionic biopolymer at a pH of about 3, 3.5, or 4 to about 4.5 or 5 has a zeta potential in a range of about −10 mV to about −55 mV, optionally wherein the anionic biopolymer at a pH of about 3, 3.5, or 4 to about 4.5 or 5 has a zeta potential in a range of about −10 mV to about −25 mV or about −35 mV to about −55 mV.

13. The complex of any preceding claim, wherein the anionic biopolymer is a polysaccharide or a glycosaminoglycan, optionally wherein the anionic biopolymer is a branched polysaccharide or a linear polysaccharide.

14. The complex of any preceding claim, wherein the anionic biopolymer is selected from a gum arabic, high methyl pectin (HMP) (optionally HMP having an esterification degree of greater than 50%), kappa-carrageenan, iota-carrageenan, dextran sulfate, sodium hyaluronate, acacia gum, xanthan gum, gellan gum, and/or a plant soluble polysaccharide (e.g., a soy soluble polysaccharide and/or a lupin soluble polysaccharide), optionally wherein the anionic biopolymer is gum arabic, acacia gum, dextran sulfate, sodium hyaluronate, and/or a plant soluble polysaccharide or the anionic biopolymer is HMP, kappa-carrageenan, iota-carrageenan, xanthan gum, and/or gellan gum.

15. The complex of any preceding claim, wherein the anionic biopolymer has a pKa of about 4 or less.

16. The complex of any preceding claim, wherein the protein, cationic biopolymer, and anionic biopolymer are each a food-grade component, optionally wherein one or more of the protein, cationic biopolymer, and anionic biopolymer are obtained and/or derived from a natural product (e.g., a food, plant, animal by-product (e.g., milk), etc.).

17. The complex of any preceding claim, wherein the protein is lactoferrin, the anionic biopolymer is a polysaccharide, and the cationic biopolymer is a gelatin, optionally wherein the anionic biopolymer is gum arabic.

18. The complex of any preceding claim, wherein the complex is a complex coacervate in a liquid, optionally wherein the complex is a multiphase coacervate in a liquid.

19. The complex of any preceding claim, wherein the complex has a coacervate-in-coacervate structure in a liquid and comprises an inner coacervate and an outer coacervate, optionally wherein the inner coacervate comprises the protein and the anionic biopolymer and the outer coacervate comprises the anionic biopolymer and the cationic biopolymer.

20. The complex of any preceding claim, wherein the complex is an interpolymeric complex.

21. The complex of any preceding claim, wherein the protein has increased stability (e.g., reduced degradation of the protein) after exposure to a temperature of about 70° C. to about 80° C., 90° C., or 100° C. for about 1 minute to about 60 minutes compared to the stability of the protein alone (i.e., not present in the complex) after exposure to the same temperature for the same period of time.

22. The complex of any preceding claim, wherein the protein, after exposure to a temperature of about 70° C. to about 80° C., 90° C., or 100° C. for about 1 minute to about 60 minutes, is degraded by less than about 30% as measured by high-performance liquid chromatography.

23. The complex of any preceding claim, wherein the activity of the protein is increased compared to the activity of the protein alone, each after storage under the same conditions (e.g., in a dried composition at about 20° C. to about 30° C. for about 1, 2, 3, 4, 5, or 6 months).

24. The complex of any preceding claim, wherein upon storage at about 4° C. to about 25° C. in a closed container for about 1, 2, 3, 4, 5, 6, 7, 8, 9, 10, 11, or 12 month(s) and/or exposure to a temperature in a range of about 70° C. or 75° C. to about 80° C., 85° C., or 90° C. for about 30 seconds to about 2 minutes or to a temperature in a range of about 100° C., 105° C., 110° C., 115° C., or 120° C. to about 125° C., 130° C., 135° C., 140° C., or 145° C. for about 2, 5, 10, 20, or 30 seconds to about 40, 50, or 60 seconds, the solubility of the complex in a composition (e.g., water, a buffer, a milk (e.g., skim milk), and/or an acid whey beverage) remains within about 30% of its original solubility prior to storage and/or the exposure.

25. The complex of any preceding claim, wherein upon storage at about 4° C. to about 25° C. in a closed container for about 1, 2, 3, 4, 5, 6, 7, 8, 9, 10, 11, or 12 month(s) and/or exposure to a temperature in a range of about 70° C. or 75° C. to about 80° C., 85° C., or 90° C. for about 30 seconds to about 2 minutes or to a temperature in a range of about 100° C., 105° C., 110° C., 115° C., or 120° C. to about 125° C., 130° C., 135° C., 140° C., or 145° C. for about 2, 5, 10, 20, or 30 seconds to about 40, 50, or 60 seconds, the amount of a biopolymer (e.g., the protein, the cationic biopolymer, and/or the anionic biopolymer) present in the complex remains within about 30% as compared to the amount of the biopolymer present in the complex prior to storage and/or the exposure.

26. The complex of any preceding claim, wherein the antimicrobial capacity and/or activity (e.g., the antibacterial activity on Gram-positive and/or Gram-negative bacteria and/or the antiviral activity) of a biopolymer (e.g., the protein, the cationic biopolymer, and/or the anionic biopolymer) present in the complex, upon storage at about 4° C. to about 25° C. in a closed container for about 1, 2, 3, 4, 5, 6, 7, 8, 9, 10, 11, or 12 month(s) and/or exposure to a temperature in a range of about 70° C. or 75° C. to about 80° C., 85° C., or 90° C. for about 30 seconds to about 2 minutes or to a temperature in a range of about 100° C., 105° C., 110° C., 115° C., or 120° C. to about 125° C., 130° C., 135° C., 140° C., or 145° C. for about 2, 5, 10, 20, or 30 seconds to about 40, 50, or 60 seconds, is retained and/or within about 30% as compared to the antimicrobial capacity and/or activity of the biopolymer prior to storage and/or the exposure.

27. The complex of any preceding claim, wherein the bioavailability of the protein is increased compared to the bioavailability of the protein alone, each after ingestion by a subject.

28. The complex of any preceding claim, wherein the complex is a particle and the anionic biopolymer and/or cationic biopolymer encapsulate the protein, optionally wherein the particle is a nanoparticle or a microparticle.

29. The complex of any preceding claim, wherein the complex in a liquid composition has an average size (e.g., diameter) of about 50 or 100 nm to about 150, 200, 300, 400, 500, 600, 700, 800, 900, 1000, 2000, 3000, 4000, 5000, or 6000 nm.

30. The complex of any preceding claim, wherein, upon storage at about 4° C. to about 25° C. in a closed container for about 1, 2, 3, 4, 5, 6, 7, 8, 9, 10, 11, or 12 month(s) and/or exposure to a temperature in a range of about 70° C. or 75° C. to about 80° C., 85° C., or 90° C. for about 30 seconds to about 2 minutes or to a temperature in a range of about 100° C., 105° C., 110° C., 115° C., or 120° C. to about 125° C., 130° C., 135° C., 140° C., or 145° C. for about 2, 5, 10, 20, or 30 seconds to about 40, 50, or 60 seconds, the size (e.g., diameter) of the complex remains within ± about 20% of its original size.

31. The complex of any preceding claim, wherein the protein, after exposure to a temperature of about 145° C. for about 2 seconds to about 60 seconds (e.g., a high-temperature short time (HTST) or an ultra-high temperature (UHT) treatment at about 145° C. for about 2 seconds to about 60 seconds), is degraded by less than 30%, optionally by less than 20%, as measured by high-performance liquid chromatography.

32. The complex of any preceding claim, wherein the antimicrobial capacity and/or activity of the protein (e.g., the antibacterial capacity and/or activity of the protein on a Gram-positive and/or Gram-negative bacteria and/or the antiviral activity), after exposure to a temperature of about 70° C. to about 80° C. or 90° C. for about 30 seconds to about 2 minutes, is retained and/or improved (e.g., increased) as compared to the antimicrobial capacity and/or activity of the protein (e.g., the antibacterial capacity and/or activity of the protein on Gram-positive and/or Gram-negative bacteria and/or the activity) alone, optionally after the same exposure conditions (e.g., the same temperature and temperature exposure time).

33. A composition comprising a complex of any one of claims 1-32, optionally wherein the composition is an aqueous composition.

34. The composition of claim 33, wherein the composition is a suspension.

35. A method of preparing a complex, the method comprising:
providing a composition comprising a protein, an anionic biopolymer, and a cationic biopolymer at a pH in a range of about 3, 3.5, or 4 to about 4.5 or 5; and
mixing the composition, thereby providing the complex.

36. The method of claim 35, wherein the protein, anionic biopolymer, and cationic biopolymer are each independently present in the composition in an amount of about 0.01%, 0.1%, 0.5%, 1%, 2%, 3%, 4%, or 5% to about 6%, 7%, 8%, 9%, or 10% by weight of the composition, optionally wherein the protein, anionic biopolymer, and cationic biopolymer are each independently present in the composition in an amount of about 0.01%, 0.1%, 0.5%, or 1% to about 2% or 3%.

37. The method of claim 35 or 36, wherein the composition comprises the anionic biopolymer and the protein in a weight ratio of about 0.5:1 to about 1:5 (anionic biopolymer:protein) and/or wherein the composition comprises the protein and the cationic biopolymer in a weight ratio of about 1:1 to about 10:1 (protein:cationic biopolymer).

38. The method of any one of claims 35-37, wherein the composition comprises the anionic biopolymer, protein, and cationic biopolymer in a weight ratio of about 1:3:1, about 4:5:1, or about 3:6:1 (anionic biopolymer:protein:cationic biopolymer).

39. The method of any one of claims 35-38, wherein mixing the composition is carried out for about 15 minutes to about 60 minutes, optionally at a temperature of about 20° C. to about 60° C.

40. The method of any one of claims 35-39, wherein providing the composition comprises forming an intermediate composition that includes the protein and the anionic biopolymer and adding the cationic biopolymer to the intermediate composition to provide the composition, optionally wherein the method further comprises mixing the intermediate composition for about 15 minutes to about 60 minutes at a temperature of about 20° C. to about 60° C.

41. The method of any one of claims 35-40, further comprising hardening the complex, optionally wherein hardening the complex comprises adjusting the temperature of the composition to about 5° C. to about 10° C. and exposing the composition to the temperature of about 5° C. to about 10° C. for about 1 hour to about 6 hours.

42. The method of any one of claims 35-41, further comprising isolating the complex from the composition, optionally wherein isolating the complex from the composition comprises centrifuging, drying, freeze-drying, and/or spray-drying the composition.

43. The method of any one of claims 35-42, wherein the total concentration of the protein, anionic biopolymer, and cationic biopolymer in the composition is about 10% by weight of the composition or less, optionally about 5% by weight of the composition or less.

44. An article comprising a complex of any one of claims 1-32, a composition of any one of claims 33-34, and/or a complex prepared according to a method of any one of claims 35-43.

45. The article of claim 44, wherein the article is a food product (e.g., infant formula, a dairy product, etc.), nutritional supplement, therapeutic drink, and/or cosmetic.
